(19) United States
(12) Patent Application Publication
(10) Pub. No.: US 2012/0283705 A1
(43) Pub. Date: Nov. 8, 2012

(54) PERCUTANEOUS DELIVERY TOOL

(75) Inventors: Brian B. Lee, Golden Valley, MN (US); Sara M. Hoeppner, Burnsville, MN (US)

(73) Assignee: MEDTRONIC, INC., Minneapolis, MN (US)

(21) Appl. No.: 13/101,613

(22) Filed: May 5, 2011

(51) Int. Cl. A61B 17/00

(52) U.S. Cl. 606/1

(57) ABSTRACT

A percutaneous delivery tool may be used to implant a medical device, for example, within a subcutaneous space. The percutaneous delivery tool may include a handle, first and second attachment members, and first and second connecting members connecting the attachment members to the handle. In some examples, the first and second attachment members are configured to receive the medical device such that the medical device is positioned between the attachment members. In some additional examples, the first and second connecting members are angled with respect to the handle, the first connecting member is angled with respect to the first attachment member, and the second connecting member is angled with respect to the second attachment member.

### TECHNICAL FIELD

This disclosure relates to implantable medical devices and, more particularly, to delivery tools for implanting medical devices.

### BACKGROUND

A variety of different medical devices may be used to monitor and/or provide therapy to a patient experiencing a medical condition. Examples of medical devices include fluid delivery devices (e.g., drug delivery devices), electrical stimulation devices, and monitoring devices. Depending on the application, these devices can be implanted through the skin of a patient to facilitate long-term monitoring and/or therapy delivery.

One type of medical device that can be implanted in a patient is a cardiac monitoring device. A cardiac monitoring device may monitor and record different conditions within a patient such as, for example, ECG signals, blood pressure, heart sounds or the like. A cardiac monitoring device can be useful for monitoring and/or diagnosing the cardiac health of the patient. For example, a cardiac monitoring device may be useful for diagnosing syncopal events and arrhythmias of the heart, which can occur infrequently and with little or no warning, making the symptoms difficult to identify and diagnose. In some examples, a cardiac monitoring device may be used to gather information about the presence or degree of various types of arrhythmias within a patient such as, for example, atrial fibrillation.

Depending on the application, a medical device such as a cardiac monitoring device may be implanted in a patient by making an incision in the patient's skin and then inserting the medical device under the skin through the incision. In some applications, a dissection tool may first be inserted through the skin to create a pocket for the medical device before subsequently implanting the medical device. In either case, a delivery tool may be used to help deliver the medical device through the incision and position the medical device under the patient's skin. However, it can be difficult to insert a medical device at precisely the right location and it can further be difficult to form a close-fitting pocket to help limit undesired motion and migration of the device after implantation.

### SUMMARY

Embodiments described herein include a delivery tool to help facilitate the implantation of a medical device through the skin of a patient. The delivery tool may be used to deliver the device into the patient during implantation. The delivery tool may also help a clinician to more easily identify a correct implantation location in the body of the patient. In some examples, the delivery tool may also allow a clinician to sense electrical (or other) signals in the body of the patient while the medical device is attached to the delivery tool. The clinician may reposition the medical device based on the detected electrical signals, e.g., to optimize signal quality.

In one embodiment, a percutaneous delivery tool includes a handle extending from a proximal end to a distal end, a first attachment member, a second attachment member, a first connecting member connecting the first attachment member to the handle, and a second connecting member connecting the second attachment member to the handle. The first attachment member and the second attachment member are configured to receive a medical device such that the medical device is positioned between the first attachment member and the second attachment member. In addition, at least one of the first attachment member and the second attachment member is movable relative to the other of the first attachment member and the second attachment member to releasably attach the medical device to the handle. In some embodiments, the first connecting member and the second connecting member are both angled with respect to the handle, the first connecting member is angled with respect to the first attachment member, and the second connecting member is angled with respect to the second attachment member.

In another embodiment, a system is described that includes a medical device and a percutaneous delivery tool. The medical device has an elongated body that defines a proximal end and a distal end. The percutaneous delivery tool includes a handle extending from a proximal end to a distal end, a first attachment member, a second attachment member, a first connecting member connecting the first attachment member to the handle, and a second connecting member connecting the second attachment member to the handle. The first attachment member and the second attachment member are configured to receive a medical device such that the medical device is positioned between the first attachment member and the second attachment member. In addition, at least one of the first attachment member and the second attachment member is movable relative to the other of the first attachment member and the second attachment member to releasably attach the medical device to the handle. In some embodiments, the first connecting member and the second connecting member are both angled with respect to the handle, the first connecting member is angled with respect to the first attachment member, and the second connecting member is angled with respect to the second attachment member.

In another example, a method is described that includes inserting a medical device having an elongated body defining a proximal end and a distal end into a patient, and releasing the medical device from a percutaneous delivery tool to which the medical device is attached. According to the example, the percutaneous delivery tool includes a handle extending from a proximal end to a distal end, a first attachment member, a second attachment member, a first connecting member connecting the first attachment member to the handle, and a second connecting member connecting the second attachment member to the handle. The first attachment member and the second attachment member are configured to receive a medical device such that the medical device is positioned between the first attachment member and the second attachment member. In addition, at least one of the first attachment member and the second attachment member is movable relative to the other of the first attachment member and the second attachment member to releasably attach the medical device to the handle. Also, the first connecting member and the second connecting member are both angled with respect to the handle, the first connecting member is angled with respect to the first attachment member, and the second connecting member is angled with respect to the second attachment member.

In an additional example, a percutaneous delivery tool includes an elongated body extending from a proximal end to a distal end, the elongated body including a substantially straight proximal portion, a substantially straight intermediate portion, and a substantially straight distal portion. According to the example, the substantially straight proximal portion is angled with respect to the substantially straight intermediate portion, and the substantially straight distal portion is angled with respect to the substantially straight intermediate portion. In addition, the substantially straight distal portion is configured to receive a medial device such that a distal portion of the medical device extends beyond the substantially straight distal portion of the elongated body.

The details of various embodiments are set forth in the accompanying drawings and the description below. Other features, objects, and advantages will be apparent from the description and drawings, and from the claims.

### BRIEF DESCRIPTION OF DRAWINGS

FIG. 1 is a conceptual diagram illustrating an embodiment including an implantable medical device and percutaneous delivery tool.

FIG. 2 is a functional block diagram illustrating components that may be included in the implantable medical device of FIG. 1

FIG. 3 is a perspective drawing illustrating a configuration that may be used for the implantable medical device of FIG. 1.

FIGS. 4 and 5 are schematic drawings illustrating side and top views, respectively, of an embodiment of a percutaneous delivery tool.

FIG. 6 is another schematic drawing illustrating a side view of the percutaneous delivery tool of FIGS. 4 and 5.

FIGS. 7 and 8 are schematic drawings illustrating top views of additional embodiments of percutaneous delivery tools.

FIGS. 9 and 10 are schematic drawings illustrating side and top views, respectively, of another embodiment of a percutaneous delivery tool.

FIG. 11 is schematic drawing illustrating a top view of the percutaneous delivery tool of FIG. 10.

FIGS. 12 are 13 are perspective drawings illustrating another embodiment of a percutaneous delivery tool.

FIG. 14 is a perspective view of an embodiment of a dissector tool.

FIG. 15 is a flow diagram illustrating an embodiment of a method for percutaneously inserting an implantable medical device using a percutaneous delivery tool according to embodiments of the invention.

FIG. 16 is conceptual drawing of an embodiment of a template that may be placed on patient to guide surgical implantation of a medical device.

FIG. 17 is schematic drawings illustrating an embodiment of the template of FIG. 16 attached to a male patient.

FIG. 18 is a schematic drawings illustrating an embodiment of the template of FIG. 16 attached to a female patient.

### DETAILED DESCRIPTION

An implantable medical device may be implanted in the body of a patient to monitor and/or treat a medical condition experienced by the patient. For example, a medical device such as a cardiac monitoring device can be implanted in the body of a patient to monitor and/or record conditions related to the cardiac health of the patient. Because the medical device is implanted, the device can monitor and record data over a long period of time without requiring patient compliance with a monitoring protocol or encumbering the patient with an external device. The data generated by the medical device may be useful to diagnose health conditions that may otherwise be difficult to detect without long-term monitoring and recording capability. However, the accuracy of the data may be dependent on the quality of the placement of the medical device during surgery. For example, if the medical device is not appropriately positioned and/or oriented within the body of the patient during implantation, noise in the signals recorded by the medical device may make it difficult to diagnose a condition of the patient. Furthermore, migration of the device after implantation can result in loss of ideal positioning. In such situations, the full diagnostic capabilities of the medical device may be underutilized.

Embodiments of the invention include a percutaneous delivery tool for implanting a medical device. The percutaneous delivery tool may releasably attach to the medical device and may facilitate placement of the medical device within a body of a patient. Depending on the application, the percutaneous delivery tool may allow a clinician to precisely place the medical device within the body of the patient. The percutaneous delivery tool may also facilitate placement of the medical device within a tight tissue pocket within the patient. Such a tight tissue pocket may help to reduce or eliminate medical device motion and migration after implantation.

For example, embodiments of the delivery tool may facilitate implantation of the medical device so that a leading edge of the medical device functions to dissect a tissue pocket in a patient during implantation. In contrast to delivery tools that fully surround and enclose a medical such as, e.g., a trocar tube or delivery needle, a delivery tool in accordance with some embodiments holds a medical device so that a leading edge of the medical device can bluntly dissect a tissue pocket as the medical device is inserted into a patient. The medical device itself may withstand the bending and/or pushing forces associated with the tunneling and dissection process, e.g., as opposed to having a protective sheath fully surrounding the tissue pocket.

By using the medical device itself to define a tissue pocket, it may be possible to maintain a tight fit between the medical device within the tissue pocket after implantation. For example, the leading edge of the medical device may dissect a tissue pocket that is equal to or substantially corresponds with the size of the medical device. By contrast, a delivery tool that fully surrounds a medical device may create a tissue pocket that is larger than the medical device, which may allow the medical device to move around within the pocket and potentially increase migration after implantation.

Embodiments of the delivery tool may have a low profile so as to minimize expansion of the tissue pocket (e.g., beyond the size of the medical device) when using the delivery tool to implant the medical device. For example, the delivery tool may include a variety of low profile and/or smooth contoured holding mechanisms to firmly hold the medical device while inserting the medical device into a patient. In some embodiments, the delivery tool is configured hold a relatively small portion of a back end of the medical device (e.g., opposite the leading edge of the medical device) to minimize tissue channel expansion and stretching when inserting the medical device.

The delivery tool may be configured to implant various types of medical devices. In some embodiments, the medical device may be a small, elongated medical device, although other sized and shaped medical devices may also be used. Independent of the specific size and shape of the medical device, the medical device may be implanted in a patient so that an axis of the medical device is parallel to skin of the patient. For example, during implantation, the medical device may be inserted at an angle relative to the skin of the patient and then rotated so that the medical device is placed parallel to the skin of the patient at a desired depth in the patient. During this process, the medical device may be pushed forward (e.g., relative to the incision location) to direct the device to a desired location.

Embodiments of the delivery tool may be shaped to facilitate precise and ergonomic implantation of the medical device. In some embodiments, the delivery tool includes two or more angled portions (e.g., two or more bends in the neck of the delivery tool) to facilitate implantation of the medical device. The two or more angled portions may provide an offset between the portion holding the medical device and the portion held by the clinician, thereby providing clearance between the clinician's hand and the surface of the patient's body throughout implantation. The two or more angled portions may also allow the clinician to controllably push and rotate the medical device through an incision in the patient, e.g., by facilitating a levering action during implantation. In instances in which the delivery tool provides an offset between the medical device and a hand of the clinician, the offset may be sized so that the medical device can be implanted at a desired depth in the patient while the handle of the delivery tool remains outside of the patient. Additional clearance may also be provided between the handle of the delivery tool and the skin of the patient so that a hand of the clinician can grip the handle of the delivery tool, e.g., in handshake style or writing style, during implantation, without the clinician's hand abutting the patient's skin.

In some embodiments, the delivery tool is also configured to hold the medical device so that an electrode (e.g., subcutaneous ECG electrode) is exposed in a tissue pocket of the patient when implanting the medical device. The electrodes may be electrodes of the medical device and/or electrodes of the delivery tool itself. In either case, a clinician may receive a signal from the electrodes while implanting the medical device, determine if the signal is appropriate (e.g., determine if the signal exhibits a suitable magnitude or quality), and, if necessary, reposition the medical device until an appropriate signal is received. In this manner, the clinician may test signal quality associated with the implant position of the medical device before the medical device is released from the delivery tool.

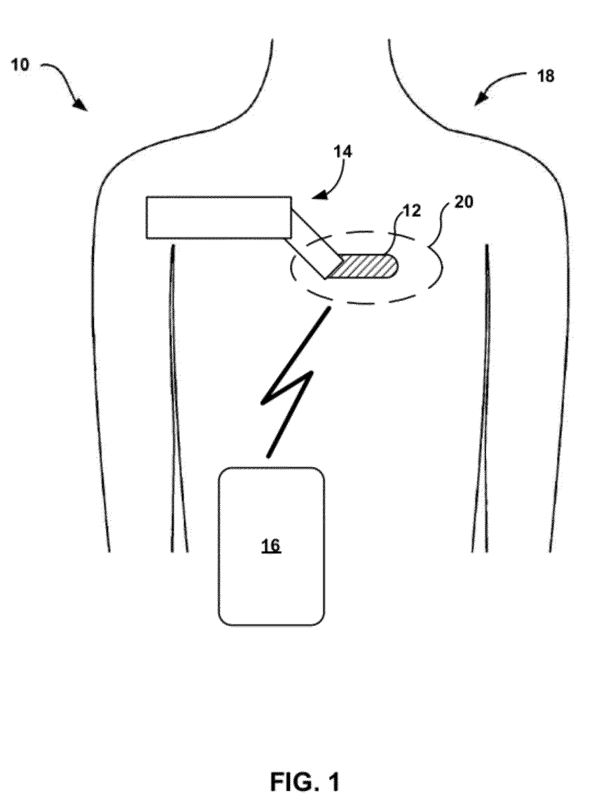

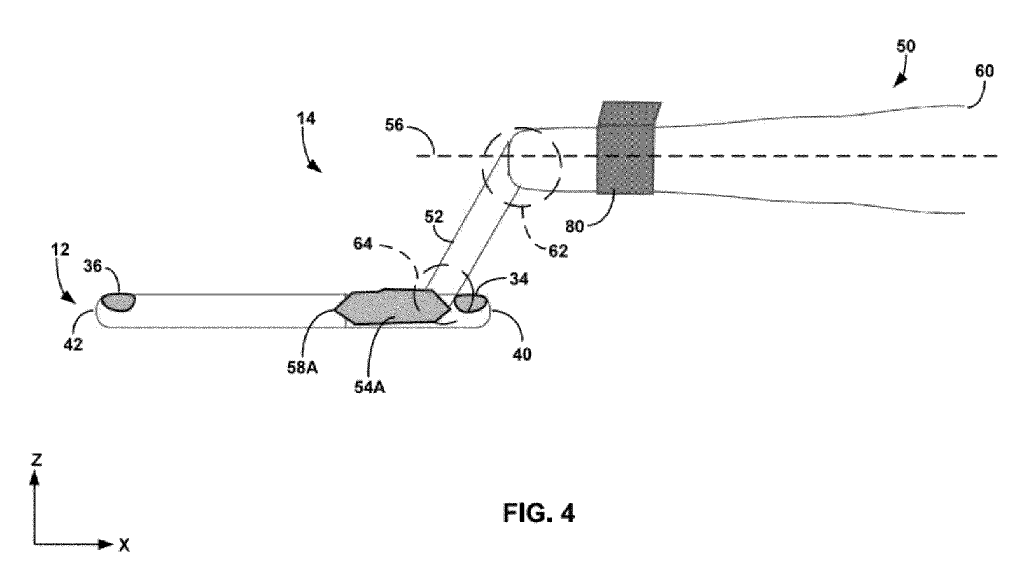

Embodiments of percutaneous delivery tools and implantation techniques will be described in greater detail with reference to FIGS. 4-14. However, an embodiment of a system that includes an implantable medical device and percutaneous delivery tool will first be described with reference to FIGS. 1-3.

FIG. 1 is a conceptual diagram illustrating a system 10, which includes an implantable medical device (IMD) 12, percutaneous delivery tool 14 (also referred to herein as “delivery tool 14”), and external programmer 16. IMD 12 may be used to monitor and/or provide therapy to a heart of patient 18. For example, IMD 12 may be an implantable monitoring device that senses electrical activity of the heart via two or more electrodes. IMD 12 may be implanted within a subcutaneous pocket relatively close to a monitoring site. For instance, in the example shown in FIG. 1, IMD 12 is implanted subcutaneously within the chest of patient 18 between the chest muscles and the outer surface of the patient's skin. In other examples, IMD 12 may be implanted within other suitable sites within patient 18, which may depend, for example, on the configuration of IMD 12 and the target monitoring and/or therapy delivery site within patient 18.

As described in greater detail below, delivery tool 14 can facilitate implantation of IMD 12 through the skin of patient 18. In some embodiments, delivery tool 14 includes a first attachment member and a second attachment member that are both disposed at a distal end of a handle. The first and second attachment members are configured to releasably attach to opposing sides of IMD 12. In such embodiments, delivery tool 14 may be used to insert IMD 12 into patient 18 so that a leading edge of IMD 12 (e.g., a distal end of IMD 12) creates a subcutaneous pocket in patient 18. Such an example implantation technique may create a tight subcutaneous pocket in patient 18 because IMD 12 itself substantially creates a pocket (e.g., in contrast to using an dissector to create the pocket). This may result in reduced IMD motion in the pocket and reduced signal degradation due to motion artifacts. In some examples, this may also reduce the change of migration of IMD 12 within the subcutaneous tissue pocket of the patient.

In other embodiments, delivery tool 14 includes a handle, at least one attachment member, and a connecting member connecting the handle to the at least one attachment member. Depending on the configuration of delivery tool 14, the delivery tool may define a first connecting angle between the handle and the connecting member and a second connecting angle between the at least one attachment member and the connecting member. The first and second connecting angles may offset the attachment member from the handle in at least two dimensions. Such an offset arrangement may allow a clinician to more easily identify an appropriate insertion depth and/or insertion position when using delivery tool 14 to insert IMD 12 into patient 18. For example, such an offset arrangement may allow a clinician to insert IMD 12 substantially parallel to the skin of the patient at a desired depth without having the clinician's hand and/or the handle of the delivery tool interfere with the insertion procedure. Delivery tool 14 can include additional or different configurations, as described in greater detail below.

IMD 12 in the example of FIG. 1 is a monitoring device configured to monitor and/or record conditions within patient 18. IMD 12 may sense electrical signals attendant to the depolarization and repolarization of a heart of patient 18 (e.g., electrogram (EGM) signals or subcutaneous electrocardiogram (ECG) signals) via electrodes (not shown in FIG. 1). For example, IMD 12 may sense electrical signals attendant to the depolarization and repolarization of a heart of patient 18 via one or more housing electrodes formed with the housing of IMD 12. In some examples, IMD 12 may sense electrical signals attendant to the depolarization and repolarization of a heart of patient 18 via electrodes coupled to one or more leads. Depending on the configuration of IMD 12, the IMD 12 may also provide electrical signals to a heart of patient 18 based on the electrical signals sensed by IMD 12. For example, IMD 12 may provide pacing pulses, cardioversion, and/or defibrillation therapy based on electrical signals sensed by IMD 12. The configurations of electrodes used by IMD 12 for sensing and/or providing electrical signals may be unipolar or bipolar.

In some examples, IMD 12 includes one or more sensors (also not shown in FIG. 1) adapted for sensing various hemodynamic conditions or other conditions of patient 18. For example, IMD 12 may include one or more blood flow sensors, blood pressure sensors, tissue perfusion sensors, pulse oximeters, hematocrit sensors, patient activity sensors, or any other sensor adapted to sense one or more hemodynamic conditions or other conditions of patient 18. The one or more sensors may be internal to a housing of IMD 12 or may be external to a housing of IMD 12 and communicatively coupled to IMD 12.

IMD 12 may be implanted at any suitable site within patient 18 using delivery tool 14. In some examples, IMD 12 is implanted within or between intra-dermal, deep dermal, or subcutaneous tissue layers of patient 18. For instance, in examples in which IMD 12 is a cardiac monitoring device, IMD 12 may be implanted between approximately 1 mm and approximately 40 mm below the surface of the skin of patient 18 such as, e.g., between approximately 5 mm and approximately 25 mm below the surface of the skin of patient 18, although other implantation depths are possible.

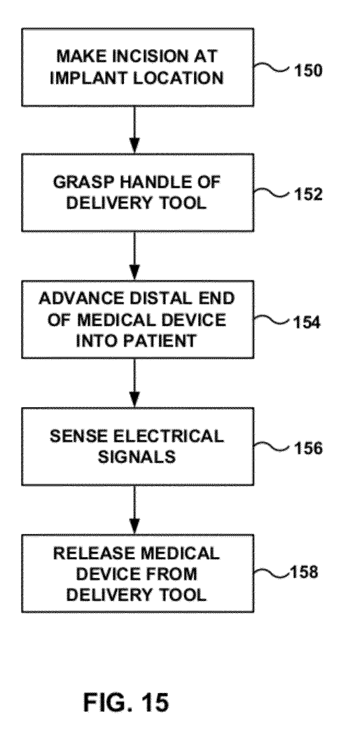

IMD 12 in the example of FIG. 1 is implanted within region 20 of patient 18. As examples, region 20 may be located on the back, on the chest, in the pelvic region, or in any other suitable region of patient 18. Further, the location of region 20 may vary based on the conditions to be monitored by IMD 12 and/or the therapy to be delivered by IMD 12. In some examples, such as examples where IMD 12 is a cardiac monitoring device, IMD 12 may be implanted in the upper chest area of patient 18 in a region proximate to the heart of patient 18. Such a location may position IMD 12 in relatively close proximity of a heart of patient 18 for receiving better (e.g., larger) electrical signals related to the cardiac health of the patient. In some examples, as described in greater detail below with respect to FIGS. 15-17, a clinician may use a body template to identify a suitable region 20 of patient 18 in which to implant IMD 12.

System 10 of FIG. 1 includes programmer 16. Programmer 16 is an external computing device that is configured to communicate with IMD 12 by wireless telemetry as needed, such as to provide or retrieve information or control aspects of IMD 12 (e.g., modify therapy or monitoring parameters, turn IMD 12 on or off, and so forth). In some examples, programmer 16 may be a clinician programmer that a clinician uses to communicate with IMD 12 and to retrieve information recorded by IMD 12. Alternatively, programmer 16 may be a patient programmer that allows patient 18 to view and modify parameters associated with IMD 12. Patient 18 may interact with programmer 16 to trigger IMD 12 to record signals monitored by the IMD, e.g., in response a fainting episode or conditions detected by the patient. The clinician programmer may include additional or alternative programming features than the patient programmer. For example, more complex or sensitive tasks may only be allowed by the clinician programmer to prevent patient 18 from making undesired or unsafe changes to the operation of IMD 12. Programmer 16 may be a handheld or other dedicated computing device, or a larger workstation or a separate application within another multi-function device.

A user such as a physician, technician, surgeon, electrophysiologist, other clinician, or patient, may interact with programmer 16 to retrieve physiological or other diagnostic information from IMD 12. For example, the user may use programmer 16 to retrieve information from IMD 12 regarding the rhythm of the heart of patient 18, trends thereof over time, conduction times of the heart, tachyarrhythmia episodes, or the like. As another example, the user may use programmer 16 to retrieve information from IMD 12 regarding sensed physiological parameters of patient 18, such as sensed electrical activity, sensed hemodynamic conditions, activity, posture, respiration, thoracic impedance, or any other conditions monitored by IMD 12. In some examples, a user may interact with programmer 16 while implanting IMD 12 to receive physiological or other diagnostic information from IMD 12. Such information may help the user determine when IMD 12 is properly positioned and, hence, properly implanted, within patient 18.

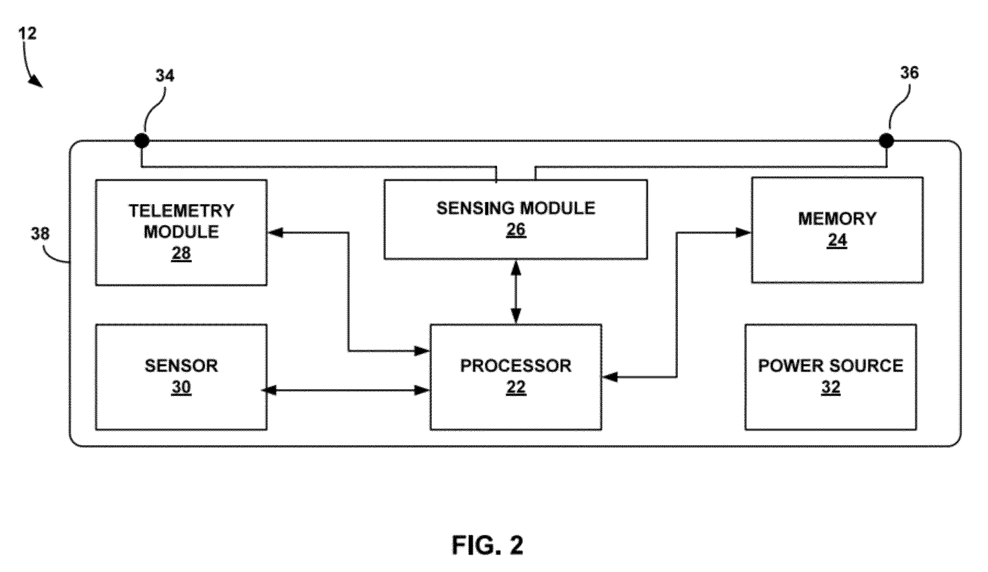

FIG. 2 is a functional block diagram of one embodiment of a configuration of IMD 12, which includes processor 22, memory 24, sensing module 26, telemetry module 28, sensor 30, and power source 32. Memory 24 may store sensed physiological parameters of patient 18 (e.g., electrogram (EGM) or subcutaneous electrocardiogram (ECG) signals). Memory 24 may also include computer-readable instructions that, when executed by processor 22, cause IMD 12 and processor 22 to perform various functions attributed to IMD 12 herein. Memory 24 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital or analog media.

Processor 22 may include any one or more of a microprocessor, a controller, a digital signal processor (DSP), an application specific integrated circuit (ASIC), a field-programmable gate array (FPGA), or equivalent discrete or integrated logic circuitry. In some examples, processor 22 may include multiple components, such as any combination of one or more microprocessors, one or more controllers, one or more DSPs, one or more ASICs, or one or more FPGAs, as well as other discrete or integrated logic circuitry. The functions attributed to processor 22 herein, as well as other processors referred to herein, may be embodied as software, firmware, hardware or any combination thereof.

Processor 22 controls sensing module 26 to sense electrical signals within patient 18, which may then be stored in memory 24. Sensing module 26 is electrically coupled to at least one electrode which, in the embodiment of FIG. 2, is illustrated as two electrodes 34 and 36. Sensing module 26 is configured to monitor electrical signals from at least one of electrodes 34 and 36 in order to monitor the electrical activity of the heart of patient 18. For example, under the control of processor 22, sensing module 26 may sense atrial events (e.g., a P-wave) and/or ventricular events (e.g., an R-wave) of the heart of patient 18. In some embodiments, sensing module 26 includes a switch module to select which of the available electrodes are used to sense the heart activity. In these examples, processor 22 can select the electrodes that function as sense electrodes, i.e., select the sensing configuration, via the switch module within sensing module 26, e.g., by providing signals via a data/address bus. In other embodiments, however, sensing module 26 does not include a switch module.

In some embodiments, sensing module 26 includes multiple detection channels, each of which may comprise an amplifier. In some embodiments, sensing module 26 or processor 22 may include an analog-to-digital converter for digitizing the signal received from a sensing channel for EGM signal processing by processor 22. In response to the sensed signals, the switch module within sensing module 26 (if present) may couple the outputs from the selected electrodes to one of the detection channels or the analog-to-digital converter.

In some embodiments, IMD 12 include one or more sensors 30 separate from electrodes 34 and 36. Via a signal generated by sensor 30, processor 22 may monitor one or more parameters of patient 18 including, e.g., a hemodynamic parameter, an activity level, and/or other conditions. Examples of sensors 30 that may generate a signal indicative of an activity level of patient 18 include an accelerometer, a bonded piezoelectric crystal, a mercury switch, or a gyro. Processor 22 may also detect one or more hemodynamic parameters via one or more sensors 30. Examples of sensors that may generate a signal indicative of a hemodynamic parameter include sensors capable of detecting heart or blood sounds, optical or ultrasonic sensors capable of detecting changes in flow associated with blood motion, or optical sensors capable of detecting oxygen saturation or tissue perfusion changes associated with blood motion.

Telemetry module 28 includes any suitable hardware, firmware, software or any combination thereof for communicating with another device, such as programmer 16 (FIG. 1). Examples of communication techniques may include, for example, low frequency or radiofrequency (RF) telemetry, but other techniques are also contemplated. Under the control of processor 22, telemetry module 28 may receive downlink telemetry from and send uplink telemetry to programmer 16 with the aid of an antenna, which may be internal and/or external. In some examples, processor 22 may transmit sensed physiological parameters produced by sensing module 26 to programmer 16. Other types of information may also be transmitted to programmer 16. Programmer 16 may interrogate IMD 12 to receive the sensed physiological parameters. Processor 22 may store the signals within memory 24, and retrieve stored signals from memory 24. Processor 22 may also generate and store marker codes indicative of different cardiac episodes that sensing module 26 detects, and transmit the marker codes to programmer 16.

The various components of IMD 12 are coupled to power source 32, which may include a rechargeable or non-rechargeable battery. A non-rechargeable battery may be selected to last for several years, while a rechargeable battery may be inductively charged from an external device, e.g., on a daily or weekly basis.

In some embodiments, as illustrated in FIG. 2, IMD 12 includes one or more housing electrodes, such as housing electrode 34 and 36. Housing electrodes 34 and 36 may be formed integrally (e.g., permanently) with an outer surface of a hermetically-sealed housing 38 of IMD 12 or otherwise coupled to housing 38. In some examples, housing electrodes 34 and 36 are defined by an uninsulated portion of an outward facing portion of housing 38 of IMD 12. Other divisions between insulated and uninsulated portions of housing 38 may be employed to define two or more housing electrodes. In some embodiments, housing electrodes 34 and 36 comprise substantially all of housing 38. Further, while IMD 12 in the example of FIG. 2 includes two housing electrodes, it should be appreciated that IMD 12 can include a different number of electrodes (e.g., one, three, four, or more) or different configuration of electrodes, and the disclosure is not limited in this respect.

For example, IMD 12 may include one or more lead electrodes in addition to or in lieu of housing electrodes 34 and 36. Lead electrodes may take the form of ring electrodes or helix tip electrodes, for example. The lead electrodes can be electrically coupled to an elongated lead body that extends from housing 38 of IMD 12. The lead electrodes may be electrically coupled to sensing module 26 and configured to sense electrical activity with the body of patient 18. Although IMD 12 may include one or more leads, in some embodiments, IMD 12 is a leadless device. A leadless IMD may be easier to implant in patient 18 than an IMD that includes lead.

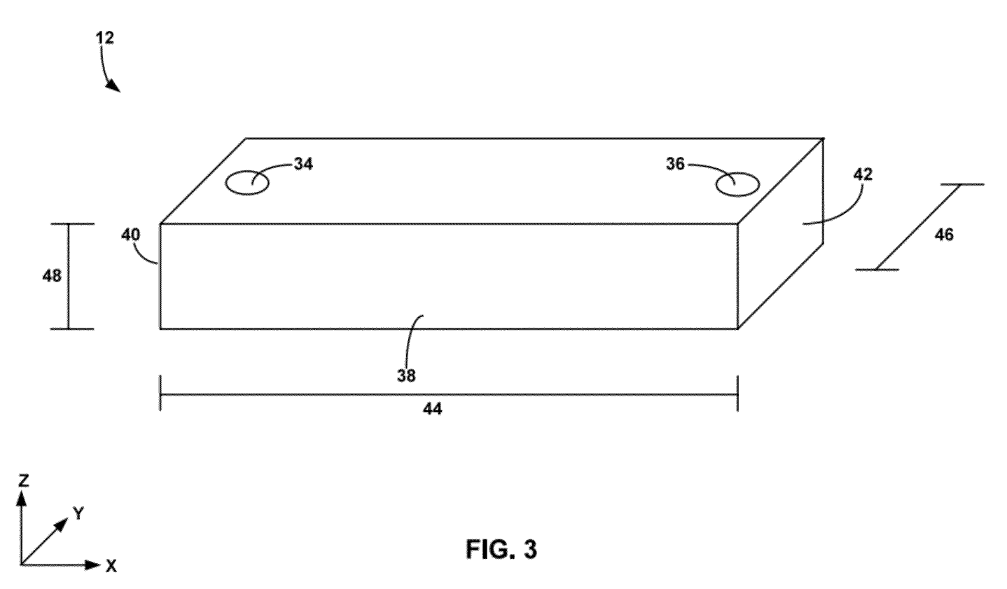

With reference to FIGS. 2 and 3, IMD 12 includes previously-described outer housing 38 that encloses various components of the IMD. Housing 38 may be constructed of a biocompatible material that resists corrosion and degradation from bodily fluids including, e.g., titanium or biologically inert polymers. In examples where IMD 12 includes housing electrode 34 and 36, the electrodes may be fabricated from any suitably electrically conductive material. Examples of electrode materials include, but are not limited to, platinum, platinum alloys, or other materials usable in implantable electrodes.

IMD 12 can define any suitable size and shape, and the size and shape of IMD 12 may vary depending on the specific configuration of IMD 12. FIG. 3 is a conceptual drawing of one embodiment of a configuration of IMD 12, which illustrates housing 38 extending from a proximal end 40 to a distal end 42. FIG. 3 also illustrates an example of an arrangement of housing electrodes 34 and 36 relative to housing 38.

In the embodiment of FIG. 3, housing 38 defines a substantially rectangular cross-sectional shape in the Y-Z, X-Z, and Y-X planes indicated on FIG. 3. In other embodiments, housing 38 may define a different polygonal (e.g., square, hexagonal) or arcuate (e.g., circular, elliptical) cross-sectional shape, or even combinations of polygonal and arcuate shapes. In some embodiments, housing 38 may define a rounded rectangular or oval shape, or housing 38 may include a rounded or curved distal end 42. Such rounded features may help a clinician to easily insert IMD 12 into patient 18 during implantation. Further, while housing 38 is illustrated as having a substantially constant cross-sectional size and shape across the a length of the housing, the size and/or shape of housing 38 may vary across the housing. For example, the cross-sectional size of housing 38 may increase (e.g., in the X-Z and/or Y-Z plane) from distal end 42 to proximal end 40. Such a housing may provide a comparatively small distal end 42 for dissecting a tissue pocket when implanting IMD 12.

Electrodes 34 and 36 can be arranged in a variety of different locations on IMD 12 including, e.g., on the same outward facing surface of housing 38. When an IMD with such a configuration is implanted into patient 18, electrodes 34 and 36 may be substantially oriented toward the skin of the patient (e.g., as opposed to the thorax of the patient). This orientation may reduce muscle movement relative to electrodes 34 and 36 as compared to other orientations, which may reduce noise sources in electrical signals sensed by sensing module 26 (FIG. 2). In other embodiments, however, electrodes 34 and 36 may be arranged on different (e.g., opposite) sides of housing 38, or at least one of electrodes 34 and 36 may be coupled to a lead extending from housing 38.

In some examples, electrodes 34 and 36 are arranged at substantially opposed ends of housing 38. For example, electrode 34 may be arranged at or adjacent to proximal end 40 of housing 38 and electrode 36 may be arranged at or adjacent to distal end 42 of housing 38. In general, increasing a separation distance between electrodes 34 and 36 may increase a single vector between the two electrodes. This may improve the signal quality of an electrical signal sensed via electrodes 34 and 36. It should be appreciated, however, that alternative electrode configurations are possible.

The specific dimensions of housing 38 of IMD 12 can vary, e.g., based on the shape of housing 38 and the size of the components encased by the housing. In some applications, IMD 12 is a comparatively large device, while in other examples, IMD 12 is designed as a comparatively small, easy to implant device. That being said, in some examples, housing 38 may define a length 44 (i.e., in the X-direction indicated on FIG. 3) between approximately 25 millimeters (mm) and approximately 65 mm such as, e.g., approximately 46 mm, a width 46 (i.e., in the Y-direction indicated on FIG. 3) between approximately 1 mm and approximately 7 mm such as, e.g., approximately 3 mm, and a height 48 (i.e., in the Z-direction indicated on FIG. 3) between approximately 3 mm and approximately 13 mm such as, e.g., approximately 8 mm. The foregoing dimensions are merely examples, however, and other dimensions are possible. In general, decreasing the size of IMD 12 may make the IMD easier to implant within patient 18. For instance, when a leading edge of IMD 12 is used to bluntly dissect a pocket within patient 18 (i.e., while IMD 12 is attached to delivery tool 14), decreasing the size of IMD 12 may make the IMD 12 easier to implant because less tissue will be dissected within patient 18.

In some examples, housing 38 of IMD 12 defines a length greater than a width and/or a height. Such an IMD may be referred to as an elongated IMD. For example, housing 38 of IMD 12 may define a length that is at least twice at long as a width or a height of the housing such as, e.g., a length that is at least three times at long as a width or a height, or a length that is at least four times as long as a length or a height. Increasing the length of IMD 12 may increase a separation distance between electrodes 34 and 36, which may improve the signal quality of an electrical signal sensed via electrodes 34 and 36.

The configuration of IMD 12 illustrated and described with respect to FIGS. 1-3 is merely one example of a medical device that can be implanted into patient 18 using delivery tool 14. Other types of medical devices may be implanted using delivery tool 14.

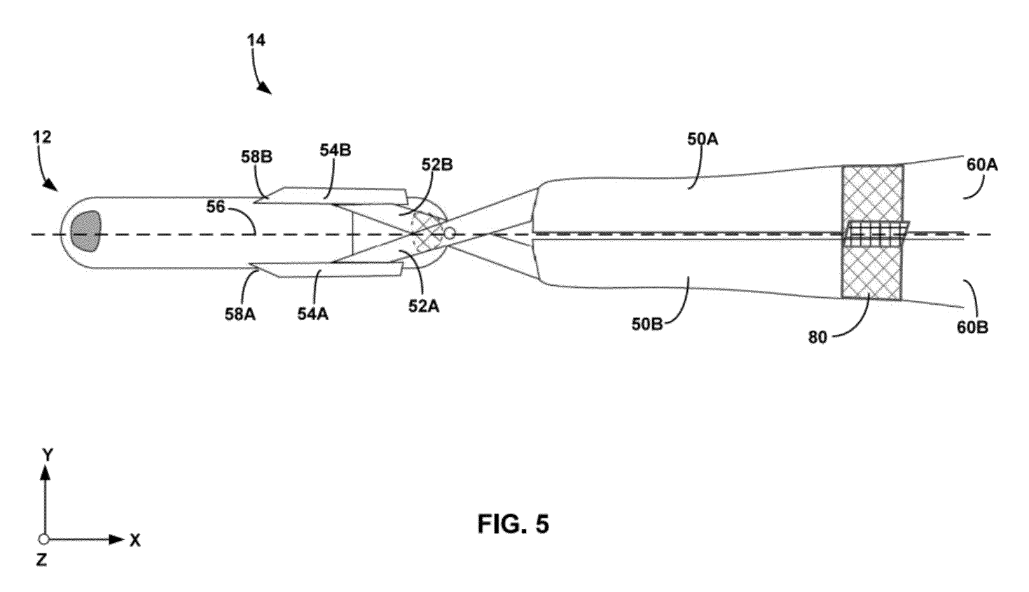

FIGS. 4 and 5 are schematic drawings illustrating side and top views, respectively, of an embodiment of a delivery tool 14. Delivery tool 14 is configured to releasably hold IMD 12 (FIGS. 1-3) for implanting the IMD into patient 18. Delivery tool 14 includes a handle 50, a connecting member 52, and at least one attachment member 54. Specifically, in the embodiment of FIGS. 4 and 5, delivery tool 14 includes a first handle member 50A and a second handle member 50B (collectively “handle 50”), a first connecting member 52A and a second connecting member 52B (collectively “connecting member 52”), and a first attachment member 54A and a second attachment member 54B (collectively “attachment member 54”). IMD 12 is positioned between first attachment member 54A and second attachment member 54B to releasably connect IMD 12 to handle 50. In use, a clinician may grip handle 50 of delivery tool 14 and manipulate the delivery tool to implant IMD 12 into patient 18.

Delivery tool 14 may be used to facilitate implantation of IMD 12 between selected layers of tissue of patient 18, although the delivery tool is not limited to such applications. Delivery tool 14 may allow a clinician to implant IMD 12 into a comparatively tight tissue pocket in patient 18. Implanting IMD 12 in a comparatively tight tissue pocket may reduce or eliminate movement of IMD 12, e.g., relative to muscle or other patient tissue, after implantation, which may reduce or eliminate noise in electrical signals sensed via electrodes 34 and 36 (FIGS. 2 and 3).

As shown in the embodiment of FIGS. 4 and 5, delivery tool 14 may connect to IMD 12 so that distal end 42 of the IMD is disposed away from first attachment member 54A and second attachment member 54B. For example, delivery tool 14 may connect to IMD 12 so that distal end 42 of IMD 12 projects away from distal ends 58A and 58B of first attachment member 54A and second attachment member 54B (e.g., in the negative X-direction indicated on FIGS. 4 and 5). In some embodiments, such as embodiments where IMD 12 defines an elongated body, delivery tool 14 may be configured to connect to a proximal portion (e.g., proximal half, proximal quarter, etc.) of IMD 12 so that a corresponding distal portion (e.g., distal half, distal three-quarters, etc.) extends away from the delivery tool (e.g., in the negative X-direction indicated on FIGS. 4 and 5).

In embodiments where distal end 42 of IMD 12 extends distally beyond the distal end of delivery tool 14, the distal end 42 of the medical device may be inserted as a leading edge through an incision in the skin of patient 18 (FIG. 1). Distal end 42 of IMD 12 may be advanced through an incision in the skin of patient 18 so that the housing of the IMD creates a pocket between layers of otherwise undisturbed tissue. In other examples, distal end 42 of IMD 12 may be advanced through a previously dissected channel where a cross-sectional area of the channel is less than a cross-sectional area of IMD 12 (e.g., in the X-Y plane indicated on FIG. 2). In either case, IMD 12 may define a pocket that substantially corresponds to the dimensions of housing 38 of the IMD. In contrast to other techniques, such as techniques that define a pocket with an introducer and then eject a medical device into the pocket through a cannula of the introducer, implanting IMD 12 with delivery tool 14 may result in a tighter pocket around the IMD after implantation.

In the example of FIGS. 4 and 5, handle 50 defines a longitudinally extending member having a major axis 56 centered about the handle 50 (i.e., when IMD 12 is attached to delivery tool 14). In some examples, first attachment member 54A and second attachment member 54B are offset from major axis 56 so that IMD 12, which is releasably held by first attachment member 54A and second attachment member 54B, is offset from major axis 56. In some embodiments, first attachment member 54A and second attachment member 54B are offset from major axis 56 in at least two dimensions. For instance, in the embodiment shown in FIGS. 4 and 5, first attachment member 54A and second attachment member 54B are offset from major axis 56 in the Z-dimension indicated on FIG. 4 and also in the Y-dimension indicated on FIG. 5. Offsetting IMD 12 from handle 50 may make it easier for a clinician to properly position IMD 12 in the body of patient 18. For example, offsetting IMD 12 from handle 50 may provide clearance between handle 50 and a patient's skin during implantation, allowing a clinician to grip the handle with minimal interference from the patient's skin. However, in other embodiments, first attachment member 54A and second attachment member 54B are not offset from major axis 56, or are offset in only one dimension.

Delivery tool 14 in the example of FIGS. 4 and 5 includes first handle member 50A connected to first attachment member 54A via first connecting member 52A, and second handle member 50B connected to second attachment member 54B via second connecting member 52B. In some examples, delivery tool 14 defines an directional change portion (e.g., an angled portion or a curved portion) or at least two directional change portions (e.g., at least two angled portions or at least two curved portions) between distal ends 58A and 58B of first connecting member 52A and second connection member 52B and proximal ends 60A and 60B (collectively “proximal end 60”) of handle members 50A and 50B. Delivery tool 14 may also define more than two directional change portions (e.g., three or more directional change portions). A directional change portion may be a portion or region of delivery tool 14 in which the tool changes orientation or direction. For instance, in the example of FIG. 4, delivery tool 14 defines a first directional change portion 62 and a second directional change portion 64.

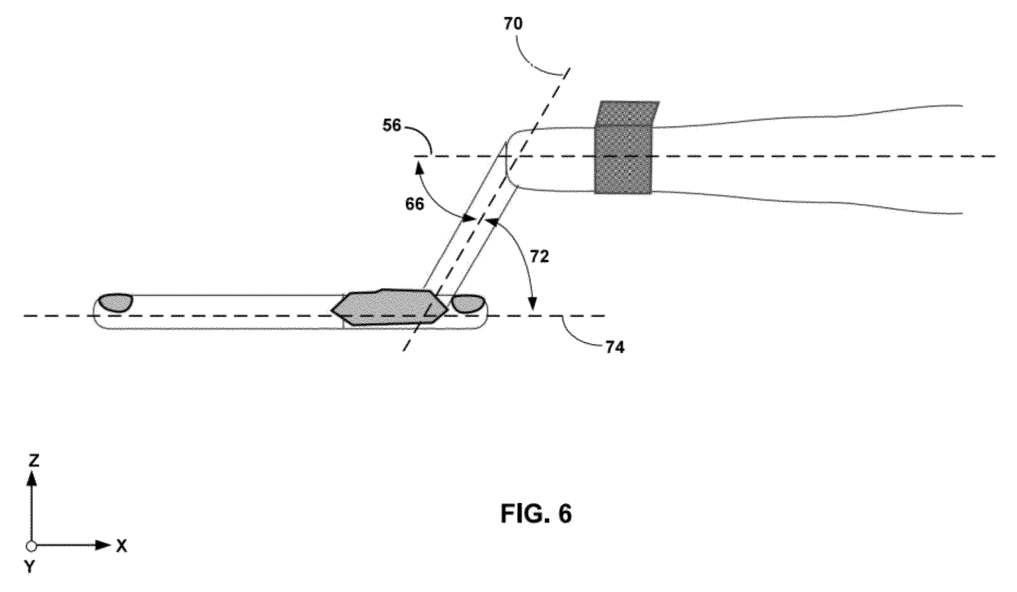

First directional change portion 62 and second directional change portion 64 are illustrated on FIG. 6, which is a reproduction of the schematic drawing of FIG. 4 where reference numerals have been removed for clarity. As shown on FIG. 6, first directional change portion 62 may be defined by a connecting angle 66 between major axis 56 extending through a handle member of delivery tool 14 and a major axis 70 extending through a connector member that is connected to the handle member. Second directional change portion 64 may be defined by a connecting angle 72 between major axis 70 extending through the connector member and a major axis 74 extending through an attachment member that is connected to the connector member. Connecting angles 66 and 72 may define any suitable angle, and each of the angles may be the same or one of the angles may be different from another of the angles. In some examples, connecting angles 66 and 72 are each non-zero angles. In various embodiments, connecting angles 66 and 72 are fixed values which may be between approximately 5 degrees and approximately 85 degrees such as, e.g., between approximately 10 degrees and approximately 80 degrees, or approximately 25 degrees and approximately 75 degrees. In some additional examples, one or both of connecting angles 66 and 72 are fixed values which are between approximately 35 degrees and approximately 55 degrees such as, e.g., approximately 45 degrees. In general, connecting angles 66 and 72 may be the same or different from each other and may be larger for applications where a deeper implant site is desired.

In some embodiments, first attachment member 54A and second attachment member 54B are positioned in a different plane(s) relative to first handle member 50A and second handle member 50B, respectively. For instance, in the example of FIG. 6, delivery tool 14 defines a first horizontal plane (i.e., X-Y plane) extending through major axis 56 of the handle member and a second horizontal plane (i.e., X-Y plane) extending through major axis 74 of the corresponding attachment member. The first and second horizontal planes may be vertically spaced from one another (i.e., in the Z-direction indicated on FIG. 6). In different configurations, the first and second horizontal planes may or may not be substantially parallel to one another.

In the embodiment of FIGS. 4 and 5, IMD 12 is releasably connected to first attachment member 54A and second attachment member 54B of delivery tool 14. In particular, IMD 12 is positioned between first attachment member 54A and second attachment member 54B of delivery tool 14 such that first attachment member 54A and second attachment member 54B are disposed against opposing sides of IMD 12. In some embodiments, as illustrated in FIG. 5, first attachment member 54A and second attachment member 54B are disposed against opposing sides of IMD 12 that do not include housing electrodes. In other embodiments, at least one of first attachment member 54A and second attachment member 54B is disposed against a side of IMD 12 that includes a housing electrode (e.g., housing electrode 34 and 36).

Independent of the specific location of first attachment member 54A and second attachment member 54B relative to IMD 12, in some examples, delivery tool 14 is configured to connect to IMD 12 so that the delivery tool 14 does not substantially block or interfere with electrical signals received by one or more housing electrodes (e.g., both housing electrodes 34 and 36) of the IMD 12. For instance, in examples in which IMD 12 includes housing electrodes 34 and 36 at or adjacent to proximal end 40 and distal end 42, respectively, of the IMD 12, delivery tool 14 may be configured to connect to the IMD without substantially enclosing proximal end 40 and/or distal end 42 of the device.

Configuring delivery tool 14 to hold IMD 12 so that the delivery tool does not substantially interfere with electrical signals received by housing electrodes of the IMD may allow a clinician may review the electrical performance of IMD 12 during implantation (e.g., while the IMD is connected to delivery tool 14). For example, during implantation, a clinician can insert IMD 12 into patient 18 (FIG. 1) and review electrical signal data from IMD 12 (e.g., via programmer 16) while the IMD is still attached to delivery tool 14 prior to detaching IMD 12. Examples of electrical signal data may include electrical cardiac signal data (e.g., the magnitude of an R-wave or an R-R interval) or other type of signal data. Based on the received data, the clinician may detach delivery tool 14 from IMD 12 or may reposition IMD 12 (e.g., deeper or shallower in the tissue layers of patient 18) until suitable electrical performance is exhibited and then may detach delivery tool 14 from IMD 12.

Delivery tool 14 includes first attachment member 54A and second attachment member 54B. First attachment member 54A and second attachment member 54B may be any feature configured to attach IMD 12 to delivery tool 14. In some examples, first attachment member 54A and second attachment member 54B have a shape that conforms to and/or corresponds with a housing shape of IMD 12. For example, first attachment member 54A and second attachment member 54B may be a pair of pinchers or jaws that are shaped to hold IMD 12. The pinchers or jaws may be low profile so as to minimize the size of a tissue pocket created with implanting IMD 12. In some examples, the pinchers or jaws have rounded or contoured distal ends to minimize tissue disruption when implanting IMD 12, although pointed or angled distal ends or other shaped distal ends are also possible.

In FIGS. 4 and 5, where IMD 12 is illustrated as defining a substantially rectangular shape, first attachment member 54A and second attachment member 54B each define at least one planar surface (e.g., in the X-Z plane indicated on FIG. 5) that can be positioned against a corresponding planar surface of IMD 12. In some additional examples, first attachment member 54A and second attachment member 54B may each define an elongated surface (e.g., in the X-direction indicated on FIG. 5) that can be positioned against an elongated body of IMD 12. First attachment member 54A and second attachment member 54B may each be sized so that each of the attachment members does not extend beyond a sidewall of a housing of IMD 12 (e.g., in the X- and/or Z-directions indicated on FIGS. 4 and 5). Such attachment members may minimize the size of a tissue pocket created by IMD 12 and delivery tool 14 when implanting the IMD. Other size and shapes of attachment members are possible though, and it should be appreciated that the disclosure is not limited in this respect.

First attachment member 54A and second attachment member 54B are connected to first connecting member 52A and second connecting member 52B, respectively. Further, first connecting member 52A and second connecting member 52B are connected to first handle member 50A and second handle member 50B, respectively. In general, first connecting member 52A and second connecting member 52B may be any feature connecting first attachment member 54A and second attachment member 54B to respective handle members. In addition, first handle member 50A and second handle member 50B may be any portion of delivery tool 14 that is configured to be gripped by a user during implantation of IMD 12. While the example delivery tool of FIGS. 4 and 5 includes two attachment members, two connecting members, and two handle members, the disclosure is not limited to the specific number or arrangement of features illustrated in the example.

For example, although the attachment members, connecting members, and handle members of delivery tool 14 are illustrated as being separate, connected features in FIGS. 4 and 5, in other embodiments, one or more of the features may be integrally (e.g., permanently) formed with one or more of the other features of the delivery tool. For example, first attachment member 54A and/or second attachment member 54B may be defined as a portion (e.g., distal portion) of first connecting member 52A and/or second connecting member 52B that is positionable against IMD 12 rather than as a separate feature extending from first connecting member 52A and/or second connecting member 52B. As another example, first handle member 50A and/or second handle member 50B may be defined as a portion (e.g., proximal portion) of first connecting member 52A and/or second connecting member 52B that can be gripped by a user rather than as a separate feature extending from first connecting member 52A and/or second connecting member 52B.

As briefly noted above, first attachment member 54A and second attachment member 54B are configured to move relative to each other to receive IMD 12 and to releasably connect the IMD to handle 50. In the embodiment shown in FIG. 5, first connecting member 52A is connected to second connecting member 52B about a central pin so that moving first handle member 50A in the positive Y-direction moves first attachment member 54A in the negative Y-direction, and moving second handle member 50B in the negative Y-direction moves second attachment member 54B in the positive Y-direction. Upon inserting IMD 12 in a space defined between first attachment member 54A and second attachment member 54B, the attachment members can be moved against IMD 12 to releasably hold the IMD. Conversely, moving first attachment member 54A and second attachment member 54B away from IMD 12 releases the IMD from delivery tool 14.

When first attachment member 54A and second attachment member 54B are positioned against IMD 12, frictional engagement between the attachment members and the IMD may prevent the IMD from moving, e.g., during implantation. Such frictional engagement may be enhanced by shape of the attachment members 54 which may be contoured to match the shape of the adjacent portion of IMD 14. For example, the attachment members 54 may be curved on the interior surface to match a curved outer surface of the IMD 12. In some embodiments, the attachment members 54 may abut IMD 12 on more than one side of the IMD 12, such that they may extend around the side of the IMD 12 to also abut a portion of the top and/or bottom surfaces of the IMD 12. Although IMD 12 may be releasably attached to delivery tool 14 via frictional engagement, the disclosure is not limited to such an example of a releasable connection. In other embodiments, other mechanical fixation features such as, e.g., clips, bolts, screws, adhesive, etc., may be used to releasably connect IMD 12 to delivery tool 14.

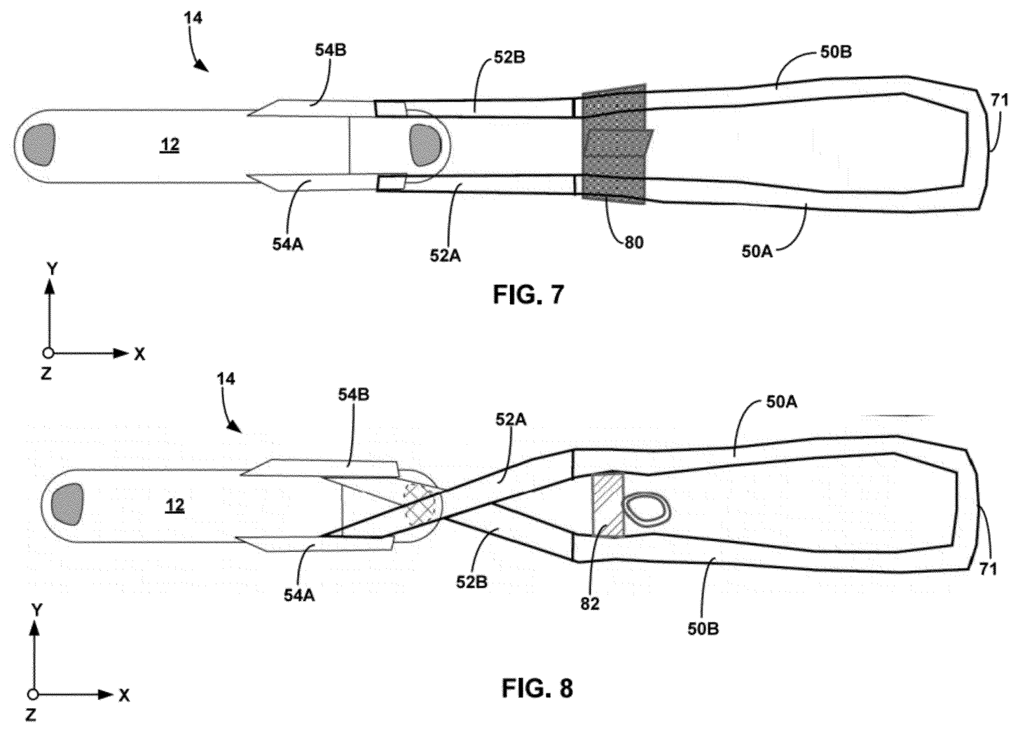

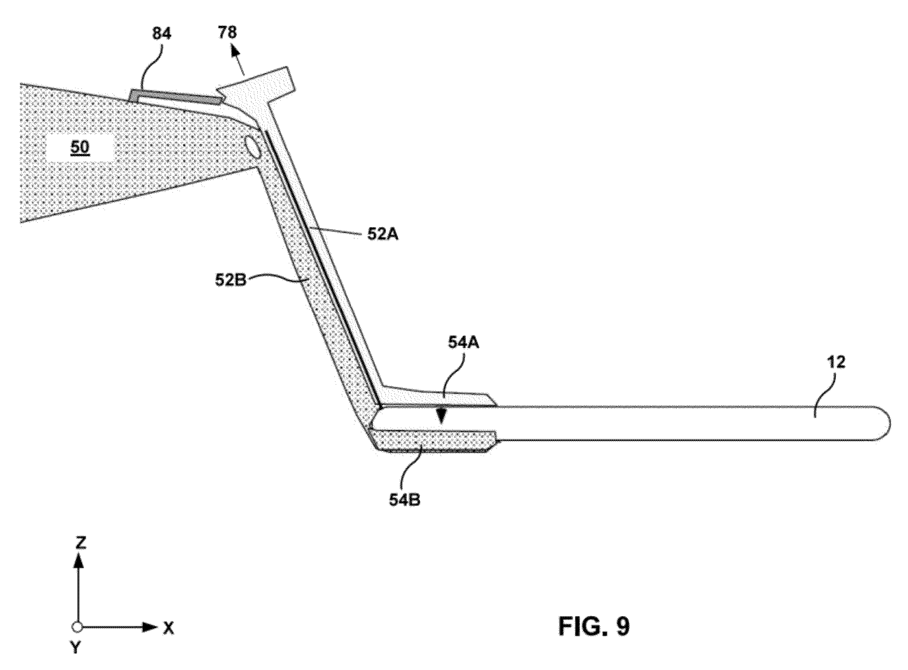

A delivery tool in accordance with the disclosure can have a number of different configurations other than the specific configuration illustrated in FIGS. 4 and 5. FIGS. 7 and 8 are schematic drawings illustrating top views of an alternative embodiment of a delivery tools 14 in accordance with the disclosure. Like reference numerals in FIGS. 7 and 8 illustrate like features describe above with respect to FIGS. 4 and 5.

With reference to FIG. 7, delivery tool 14 includes previously described first and second handle members 50A and 50B, first and second connecting members 52A and 52B, and first and second attachment members 54A and 54B. Unlike delivery tool 14 in the example of FIGS. 4 and 5, however, first and second handle members 50A and 50B are joined at a proximal end 71 to define a unitary handle structure. Further, first and second connecting members 52A and 52B are not joined about a central pin as in the example of FIG. 5. Rather, first and second connecting members 52A and 52B extend along opposing sides of IMD 12 so that moving first handle member 50A in the positive Y-direction moves first attachment member 54A in the positive Y-direction, and moving second handle member 50B in the negative Y-direction moves second attachment member 54B in the negative Y-direction. In some embodiments, a distance between first connecting member 52A and second connecting member 52B (i.e., in the Y-direction indicated on FIG. 7) is less than a width of IMD 12 so that first and second attachment members 54A and 54B bias towards IMD 12 when IMD 12 is positioned between the attachment members 54A and 54B. In such a configuration, delivery 14 may apply a compressive force against IMD 12 (when IMD 12 is positioned between first and second attachment members 54A and 54B) without user assistance. In other embodiments, the distance between first connecting member 52A and second connecting member 52B is greater than a width of IMD 12 so that the IMD is released from delivery tool 14 unless first handle member 50A and second handle member 50B are physically held in position biased towards each other. In such a configuration, delivery 14 may apply a compressive force against IMD 12 (when IMD 12 is positioned between first and second attachment members 54A and 54B) when a user compresses first handle member 50A and second handle member 50B together.

With reference to FIG. 8, delivery tool 14 also includes previously described first and second handle members 50A and 50B, first and second connecting members 52A and 52B, and first and second attachment members 54A and 54B. In contrast to delivery tool 14 in the example of FIGS. 4 and 5, first and second handle members 50A and 50B are joined at a proximal end 71 to define a unitary handle structure. Further, first and second attachment members 54A and 54B in the example of FIG. 8 bias towards IMD 12 when the IMD is positioned between the attachment members. That is, first and second attachment members 54A and 54B are configured to apply a compressive force against IMD 12 without requiring a user to compress first and second handle members 50A and 50B. To release IMD 12 from delivery tool 14, first and second handle members 50A and 50B are moved (e.g., compressed) toward each other (e.g., in opposing Y-directions) to move first attachment member 54A in the negative Y-direction and second attachment member 54B in the positive Y-direction.

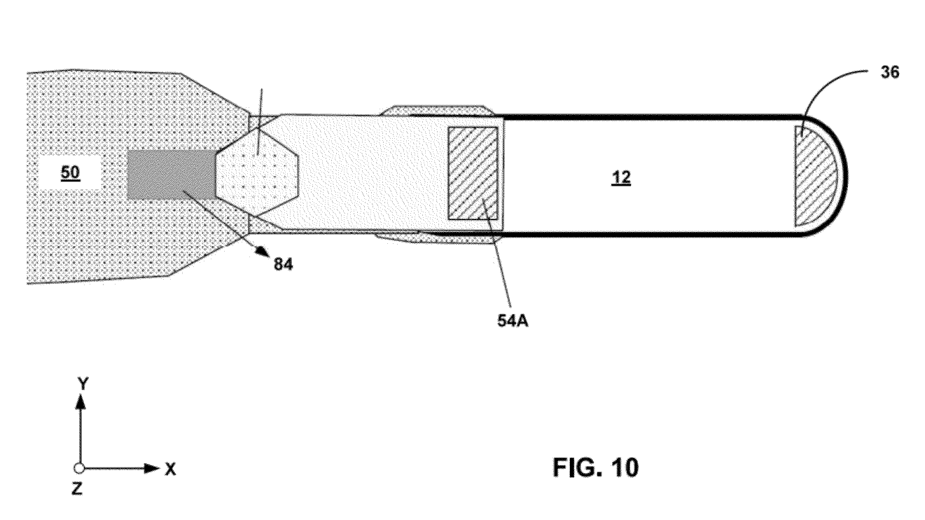

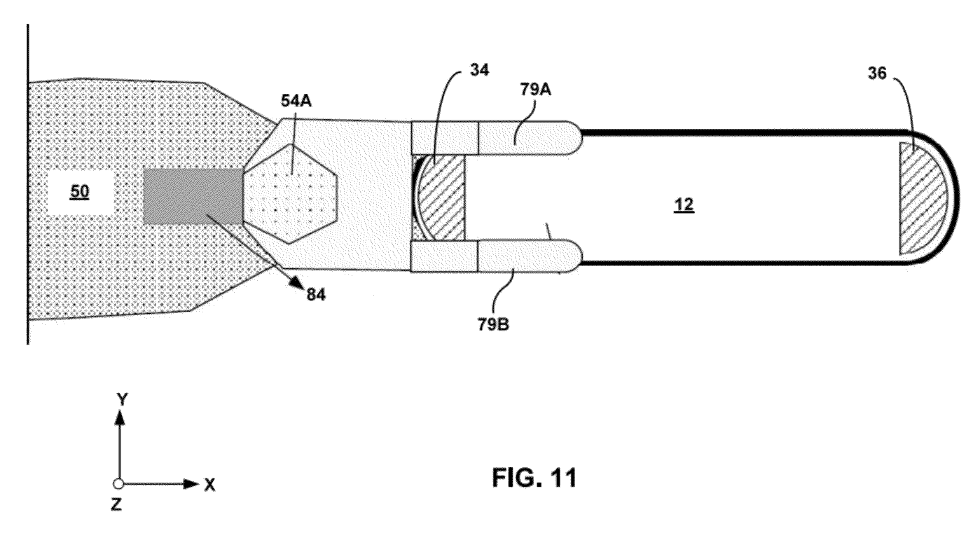

FIGS. 9 and 10 are schematic drawings illustrating side and top views, respectively, of another alternative embodiment of a delivery tool 14 in accordance with the disclosure. Like reference numerals in FIGS. 9 and 10 illustrate like features describe above with respect to FIGS. 4 and 5.

With reference to FIGS. 9 and 10, delivery tool 14 includes a handle 50 that comprises a single handle member connected to a first connecting member 52A and a second connecting member 52B. First connecting member 52A is connected to first attachment member 54A, while second connecting member 52B is connected to second attachment member 54B. When delivery tool 14 is connected to IMD 12 as illustrated in FIGS. 9 and 10, first attachment member 54A and second attachment member 54B are disposed adjacent opposed surfaces of the IMD in the X-Y plane. In some embodiments, at least one of these surfaces of IMD 12 includes a housing electrode (e.g., housing electrode 34 or 36). In addition, when delivery tool 14 is configured as illustrated in FIGS. 9 and 10, delivery tool 14 is connected to IMD 12 so that a proximal portion of the IMD is frictionally secured between first attachment member 54A and second attachment member 54B and a distal portion of the IMD extends away from the delivery tool. IMD 12 may be released from delivery tool 14 by moving first attachment member 54A away from second attachment member 54B in the direction indicated by arrow 78.

In some embodiments, as illustrated in FIG. 10, first attachment member 54A may substantially cover a top portion of IMD 12 that includes one or more housing electrodes. Such an example attachment member may provide increased surface area for frictionally engaging with a corresponding surface of the IMD, thereby securely connecting the IMD to the delivery tool. In other embodiments, however, first attachment member 54A may be configured so that the attachment member does not substantially block or interfere with electrical signals receive by one or more housing electrodes (e.g., both housing electrodes 34 and 36) of IMD 12.

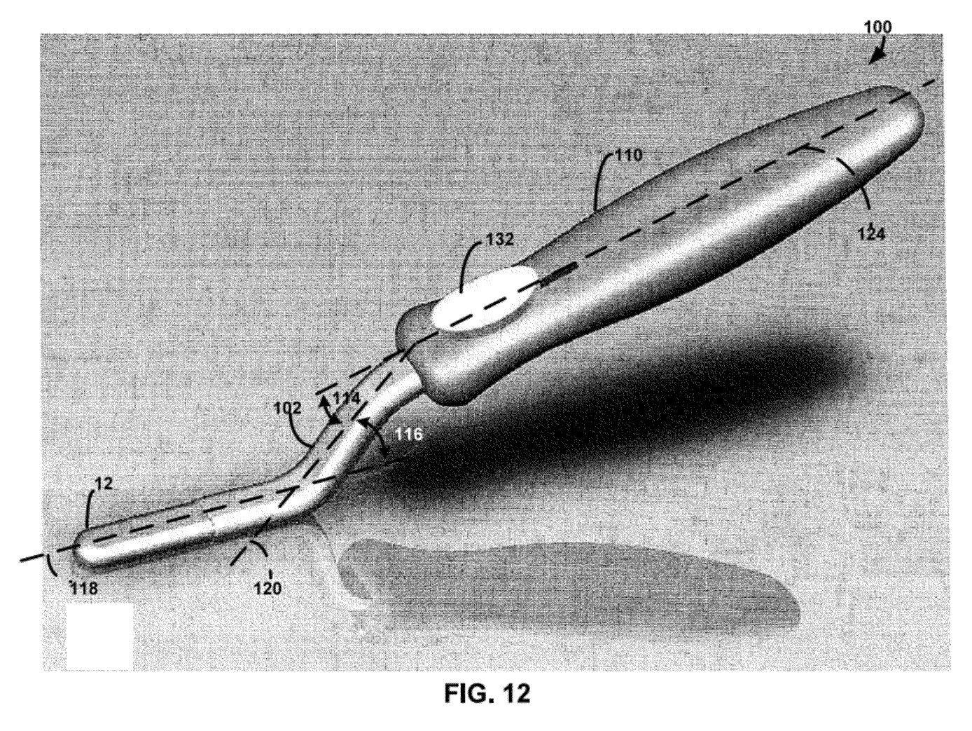

FIG. 11 is a schematic drawing illustrating a top view of an alternative embodiment of an attachment member that may be used with delivery tool 14. In the example of FIG. 11, attachment member 54 defines two clamping members 79A and 79B that project distally from an attachment member base. A cutout is defined between clamping members 79A and 79B that is configured (e.g., sized and positioned) to receive housing electrode 34 so that the housing electrode is not substantially blocked by attachment member 54A.

A delivery tool in accordance with the disclosure can include features in addition to or in lieu of those features described above with respect to FIGS. 4-11. As one example, a delivery tool in accordance with the disclosure can include a locking member affixed to a handle to lock different attachment members in a substantially fixed position relative to one another. FIGS. 4-7 illustrate embodiments of a locking mechanism 80 wrapped around first handle member 50A and second handle member 50B. Locking mechanism 80 may be a band of material such as, e.g., plastic, metal wire, or paper. FIG. 8 illustrates another embodiment of a locking mechanism 82 positioned between first handle member 50A and second handle member 50B. Locking mechanism 82 may be formed from a substantially rigid material that biases the handle members away from one another. FIGS. 9-11 illustrate yet another embodiment of a locking mechanism 84. Locking mechanism 84 may be a depressible catch feature that fits into a corresponding notch on first connecting member 52A.

Locking mechanism 80, 82, and 84 may function to bias handle members 50A and 50B of delivery tool 14 against IMD 12. Locking mechanism 80, 82, and 84 may also prevent first attachment member 54A from moving relative to second attachment member 54B. Locking mechanism 80, 82, and 84 may be useful in instances when IMD 12 and delivery tool 14 are provided as a preassembled kit, e.g., for storage and shipping. In some examples, locking mechanism 80, 82, and 84 may be moveable (e.g., removable) upon application of hand pressure without requiring the use of a tool (e.g., a cutting tool).

As another example of a feature that may be included with delivery tool 14, the delivery tool may include one or more test electrodes positioned on a portion of tool that is inserted into patient 18 during implantation. In such an example, the delivery tool may also include associated circuitry and operating hardware or software for operating the electrodes. The one or more test electrodes may function similar to electrodes 34 and 36 described above with respect to IMD 12 (FIGS. 2 and 3). Further, the circuitry and operating hardware or software of delivery tool 14 may function similar to circuitry and operating hardware or software described above with respect to IMD 12. Depending on the configuration of delivery tool 14, the delivery tool may communicate signals sensed by the one or more test electrodes to a computing device positioned outside of the body of patient 18 using wired or wireless communication techniques.

In instances in which delivery tool 14 includes one or more test electrodes, a clinician may receive data sensed via the one or more electrodes while using the delivery tool to implant IMD 12. The data may provide an indication of whether IMD 12 is positioned at an appropriate location within a body of patient 18 or whether the IMD 12 should be repositioned (e.g., to a deeper or shallower depth). Delivery tool 14 may include one or more test electrodes for implanting an IMD that does not include electrodes, although the delivery tool is not limited to such an application.

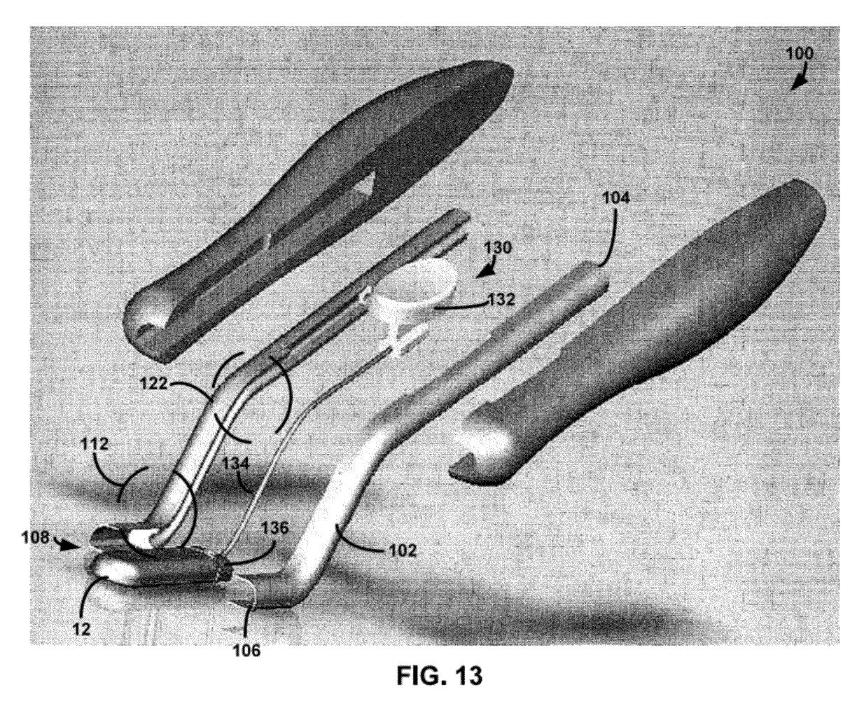

FIGS. 12 and 13 are conceptual perspective views of another embodiment of a delivery tool 100 in accordance with the disclosure. FIG. 12 is a perspective view of delivery tool 100 when the delivery tool is assembled and connected to IMD 12. FIG. 13 is a perspective view of delivery tool 100 showing different constituent parts of the delivery tool.

Delivery tool 100 is configured to releasably hold IMD 12 (FIGS. 1-3) for implanting the IMD into patient 18. Delivery tool 100 includes an elongated body 102 that extends from a proximal end 104 to a distal end 106. Elongated body 102 defines a cavity 108 at distal end 106 of the delivery tool. Cavity 108 is configured (e.g., sized and shaped) to receive IMD 12 to releasably connect the IMD 12 to the delivery tool. Specifically, in the example of FIGS. 12 and 13, cavity 108 is configured to receive a proximal portion of an elongated IMD 12 so that a distal portion of the IMD projects away from the delivery tool 100 when assembled. In some examples, a handle cover 110 extends around a handle portion of elongated body 102 to help a clinician grip the delivery tool 100 during implantation of IMD 12.

In addition, delivery tool 100 includes at least one directional change portion which, in the example of FIGS. 12 and 13, is illustrated as two directional change portions 112 and 122. As described above with respect to FIGS. 4 and 5, a directional change portion may be a portion or region of a delivery tool in which the tool changes orientation or direction. A directional change portion may be useful to help a clinician more easily find a correct implant location (e.g., tissue depth) when implanting IMD 12.

As shown in FIG. 12, a first directional change portion 112 may be defined by a connecting angle 116 between a major axis 118 extending through a substantially straight distal portion of elongated body 102 and a major axis 120 extending through a substantially straight intermediate portion of elongated body 102. A second directional change portion 122 may be defined by a connecting angle 114 between major axis 120 extending through a substantially straight intermediate portion of elongated body 102 and a major axis 124 extending through a substantially straight proximal portion of elongated body 110. Connecting angles 114 and 116 are fixed values which vary for different embodiments and may define any suitable angle including, e.g., those angles discussed above with respect to connecting angles 72 and 66, respectively.

Delivery tool 100 is configured to receive IMD 12 so that a distal portion of the IMD 12 projects away from (distally from) the delivery tool 100 when assembled. In various examples, delivery tool 100 may be configured to receive IMD 12 so that at least a distal quarter of the IMD 12 projects away from distal end 106 of delivery tool 100 such as, e.g., at least a distal half of the IMD 12 projects away from delivery tool 100, or at least a distal three-quarters of the IMD 12 projects away from delivery tool 100. In other examples, delivery tool 100 may be configured to receive IMD 12 so that at least a proximal quarter of the IMD projects into cavity 108 of the delivery tool 100 to releasably secure the IMD 12 to the delivery tool 100.

In some examples, delivery tool 100 includes a release mechanism for releasing IMD 12 from the delivery tool. Delivery tool 100 can include any suitable release mechanism for controllably releasing IMD 12 from the delivery tool. In the example of FIGS. 12 and 13, delivery tool 100 includes release mechanism 130, which includes a release button 132, a connection rod 134, and an IMD discharging member 136. IMD discharging member 136 is configured (e.g., sized and shaped) to advance through cavity 108 of delivery tool 100. Connection rod 134 extends through at least a portion of elongated body 102 to connect release button 132 to IMD discharging member 136. In operation, release button 132 is advanced parallel to elongated body 102 to advance IMD discharging member 136 through cavity 108. IMD discharging member 136 contacts a proximal end of IMD 12 and advances the IMD 12 out of cavity 108. In this manner, release mechanism 130 may function to release IMD 12 from delivery tool 100.

While delivery tools 14 and 100 have been described as being configured to hold IMD 12 for inserting the IMD into a body of patient 18, in other embodiments, a tool in accordance with the disclosure may be a tissue dissector tool rather than a medical device delivery tool. A tissue dissector tool may be used to dissect a tissue pocket in patient 18 prior to inserting IMD 12 into the tissue pocket. Such dissector tools may have dimensions and shapes corresponding to delivery tools 14 and 100, as described above.

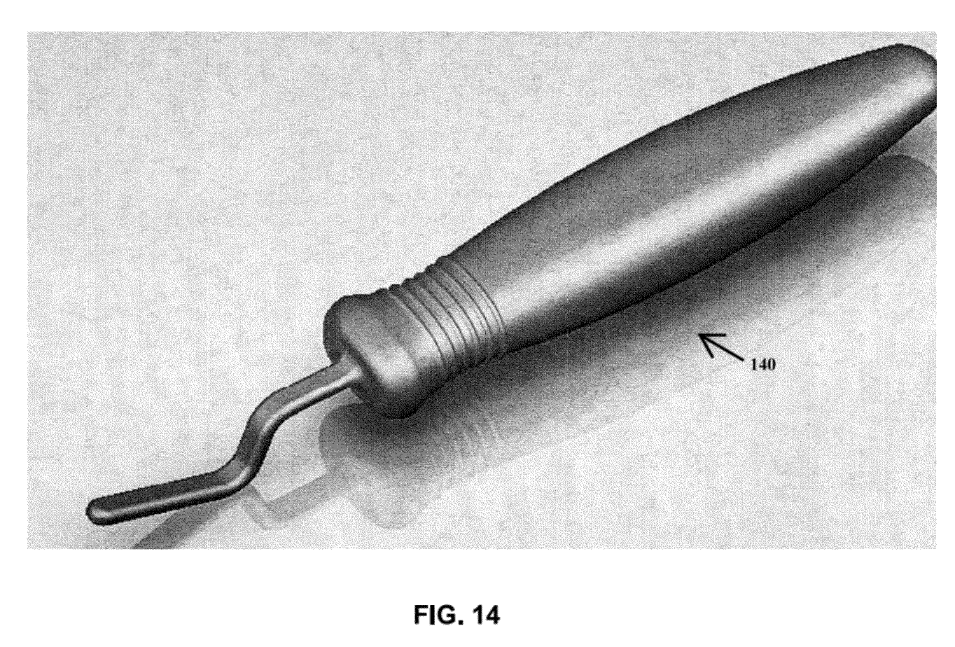

FIG. 14 is a conceptual perspective view of an embodiment of a dissector tool 140 in accordance with the disclosure. The illustrated dissector tool 140 is shaped and sized as described above with respect to delivery tool 100. However, unlike delivery tool 100 which includes cavity 108 that is configured to receive a proximal portion of an elongated IMD 12, the illustrated dissector tool 140 has an integrally (e.g., permanently) formed distal projection that is configured to dissect a tissue pocket in patient 18. The integral distal projection may correspond to a size and shape of the housing of IMD 12 (e.g., when IMD 12 is connected to delivery tool 100) so that the dissector tool 140 has dimensions that correspond to an assembled delivery tool/medical device system. It may be preferable that this integral distal projection be somewhat smaller in some or all dimensions, but similar in shape, so that when the IMD is placed in the prepared tissue pocket, the fit will be tight. As with delivery tool 100, a dissector tool 140 may have one or more directional change portions, as illustrated in FIG. 14. A directional change portion may be useful to help a clinician more easily find a correct implant location (e.g., tissue depth) when creating a tissue pocket before implanting IMD 12.

FIG. 15 is a flow diagram illustrating embodiments of methods for percutaneously inserting a medical device using a percutaneous delivery tool in accordance with the disclosure. For ease of description, the method of FIG. 15 will be described with respect to delivery tool 14 of FIGS. 4-6 and IMD 12 of FIGS. 1-3. In other embodiments, however, the method of FIG. 15 may be performed using delivery tools with different configurations or different types of medical devices, as described herein.

Initially, a clinician makes an incision at a desired implant location (150). The implant location may be any region of the body of patient 18. In some examples, the implant location is in the chest region of patient 18. For example, the implant location may be in the upper chest regions of patient 18. The incision can be of any suitable length and the length and may vary, e.g., based on the size of IMD 12. In some embodiments, the incision is between approximately 5 mm long and approximately 15 mm long such as, e.g., approximately 10 mm long.

After making an incision (150), the clinician grasps delivery tool 14 around first handle member 50A and second handle member 50B (152). In some examples, IMD 12 is pre-attached to delivery tool 14. In other examples, the clinician positions IMD 12 between first attachment member 54A and second attachment member 54B and closes the attachment members about IMD 12 to frictionally secure the IMD to delivery tool 14. Depending on the configuration of delivery tool 14, the clinician may also remove locking mechanism 80 before or after grasping first handle member 50A and second handle member 50B.

While holding handle 50 of delivery tool 14, the clinician advances distal end 42 of IMD 12 into the incision in patient 18 (154). The clinician may advance distal end 42 of IMD 12 into patient 18 so that major axis 74 of IMD 12 and/or major axis 56 of handle 50 are angled with respect to the skin of patient 18. In some examples, the clinician initially advances distal end 42 of IMD 12 into patient 18 so that major axis 74 of IMD 12 and/or major axis 56 of handle 50 are angled between approximately 90 degrees (e.g., substantially perpendicular) and approximately 20 degrees with respect to the skin of patient 18. Thereafter, once the IMD 12 has been inserted to the desired depth, the clinician may turn (e.g., rotate while pushing forward) handle 50 until major axis 74 of IMD 12 and/or major axis 56 of handle 50 are substantially parallel with the skin of patient 18. The clinician may then advance the IMD 12 forward through the tissue along an approximately linear pathway at a constant tissue depth by moving the handle 50 forward. This motion is facilitated by the shape of the delivery tool 14 having an offset handle 50 approximately parallel to the major axis 74 of the IMD 12. These motions may position IMD 12 in a tight tissue pocket. Regardless of the angle of entry into patient 18, the clinician may advance IMD 12 to a desired depth of implant in patient 18. In some examples, the clinician may implant IMD 12 between approximately 1 mm and approximately 40 mm (or more for heavier patients) below the surface of the skin of patient 18. In some examples, the clinician implants IMD 12 so that the IMD does not penetrate muscle or muscle fascia of patient 18. For example, the clinician may implant IMD 12 so that the IMD is on the surface of a muscle fascia underlying a subdermal fat layer of patient 18.

With IMD 12 initially positioned at a desired implant site with patient 18, the clinician may sense electrical signals within patient 18, e.g., via housing electrodes 34 and 36 (156) or, in alternative embodiments, via test electrodes on the delivery to 0114. The clinician may interact with programmer 16 to review electrical data sensed by sensing module 26. Based on the sensed signals, the clinician may determine that IMD 12 is suitably implanted in patient 18. Alternatively, the clinician may reposition IMD 12 (e.g., deeper or shallower in the tissue layers of patient 18 or further forward or back) and again sense electrical signals within patient 18. The clinician can continue to reposition IMD 12 until the clinician is satisfied, e.g., with the electrical performance of the IMD.

After suitably positioning IMD 12 in patient 18, the clinician releases IMD 12 from delivery tool 14 (158). The clinician moves first handle member 50A and second handle member 50B in opposing directions to move first attachment member 54A and second attachment member 54B away from IMD 12. In alternative embodiments, IMD 12 may be released by discharging it from the device 14, such as by pushing it forward using a discharging member 136. The clinician then extracts delivery tool 14 from patient 18.

As noted above, IMD 12 can be implanted in any suitable location and at any suitable depth within the body of patient 18. Further, a clinician may use any acceptable techniques to identify a suitable location and depth for implanting IMD 12. In accordance with embodiments of the disclosure, a clinician may use a body template to identify a suitable implant location and/or implant depth for implanting IMD 15. A body template may be an easy-to-use surface anatomy-cued template that shows a desirable location for implanting IMD 12 in order to provide a desired signal and/or signal quality from IMD 12 for a pre-determined parameter that IMD 12 is configured to monitor.

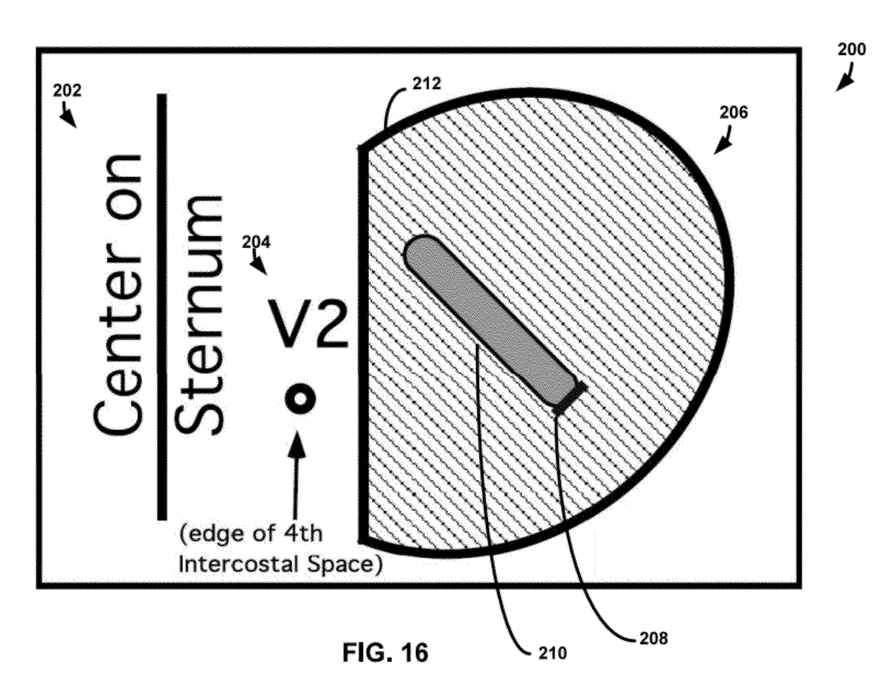

FIG. 16 is conceptual drawing of an example body template 200 that a clinician may place on patient 18 to help guide the implantation of IMD 12. Template 200 includes at least one anatomical indicator for orienting template 200 on patient 18 which, in the example of FIG. 16, is illustrated as a first anatomical indicator 202 and a second anatomical indicator 204. Template 200 also includes at least one medical device positioning indicator 206. In use, a clinician places template 200 over the body of patient 18 so that first anatomical indicator 202 and second anatomical indicator 204 are aligned with corresponding anatomical landmarks on patient 18. The clinician then uses medical device positioning indicator 206 as a reference point for implanting IMD 12 into the body of patient 18. In contrast to ad hoc or idiosyncratic implantation approaches, body template 200 may allow a clinician to quickly and precisely implant IMD 12 in a location of patient 18 that is predetermined to provide satisfactory medical device performance.

First anatomical indicator 202 and second anatomical indicator 204 may be any markings on template 200 that correspond to anatomical points on the body of patient 18 for positioning template 200. For example, first anatomical indicator 202 and second anatomical indicator 204 may be words, structures, diagrams, or combinations thereof, or other indicators that indicate where a clinician should place template 200 on the body of patient 18. In some examples, template 200 includes at least one anatomical indicator for aligning template 200 in a first dimension on the body of patient 18 and at least one other anatomical indicator for aligning template 200 in a second dimension different than the first dimension on the body of patient 18. For instance, template 200 may include an anatomical indicator for longitudinally aligning template 200 on the body of patient 18 and another anatomical indicator for latitudinally aligning template 200 on the body of patient 18. Such a combination of indicia can facilitate precise placement of template 200 on the surface of the skin of patient 18.

In the example of FIG. 16, first anatomical indicator 202 includes a vertical line and the words “Center on Sternum.” First anatomical indicator 202 instructs the clinician to center the vertical line on the sternum of patient 18. Second anatomical indicator 204 in the example of FIG. 16 includes a circle, the term “V2,” and the words “edge of 4^{th} intercostal space.” Second anatomical indicator 204 instructs the clinician to place the circle on the sternal edge of the center of the 4^{th} intercostal space of patient 18, in a position typically associated with the V2 electrode of a twelve lead system. The sternum and 4^{th} intercostal space of patient 18 are two example anatomical points on the body of patient 18 that can be used to align template 200. Such anatomical points may be useful for orienting medical device positioning indicator 206 in the upper chest region of patient 18. As noted above, implanting IMD 12 in the upper chest region of patient 18 may position IMD 12 in relatively close proximity of a heart of the patient 18 for receiving better (e.g., larger) electrical signals related to the cardiac health of the patient. In other examples, however, template 200 may include indicia that reference additional or different anatomical points on patient 18 and the anatomical points may vary, e.g., based on the type of medical device template 200 is configured to be used with.

In some examples, one or both of first anatomical indicator 202 and second anatomical indicator 204 may be independent of a specific body type. That is, first anatomical indicator 202 and second anatomical indicator 204 may include universal indicators that are substantially independent of the specific anatomy (e.g., patient gender, patient size, etc.) of a patient. Example universal indicators may include, but are not limited to, the sternum of a patient, a V2 electrode location on a patient that corresponds to a V2 electrode of a twelve lead system, and a V3 electrode location on a patient that corresponds to a V3 electrode of a twelve lead system.

In other examples, one or both of first anatomical indicator 202 and second anatomical indicator 204 may be associated with a particular body type. That is, first anatomical indicator 202 and second anatomical indicator 204 may include indicators that correspond to anatomical points on a patient of a specific size, gender, etc. In such an example, template 200 may be part of a set of templates where different templates have different anatomical indicia associated with different body types. Alternatively, template 200 may include multiple sets of anatomical indicia, where different sets of anatomical indicia are associated with different body types. Further, while template 200 in the example of FIG. 16 includes first anatomical indicator 202 and second anatomical indicator 204, it should be appreciated that a template in accordance with the disclosure may include fewer anatomical indicia (e.g., a single indicator) or more anatomical indicia (e.g., three, four, or more indicia) and the disclosure is not limited in this respect.

Template 200 includes medical device positioning indicator 206. Medical device positioning indicator 206 indicates where and/or how a clinician should position IMD 12 on and/or in the body of patient 18. Medical device positioning indicator 206 may be implemented as words, structures, diagrams, or combinations thereof, or any other suitable indicator.

In some examples, medical device positioning indicator 206 includes an incision indicator that indicates where a clinician should make an incision in the skin of patient 18 for implanting IMD 12. For instance, in the example of FIG. 16, template 200 includes incision indicator 208, which is a line that a clinician can cut along to create an incision in the skin of patient 18. In some examples, incision indicator 208 may be positioned on template 200 so that a clinician makes an incision on patient 18 that is calibrated for a specific delivery tool such as, e.g., delivery tool 14. For instance, for a delivery tool with a specific neck length, incision indicator 208 may be positioned on template 200 so that a clinician makes an incision that accounts for the length of a tunnel that the delivery tool creates during implantation.

In some additional examples, medical device positioning indicator 206 includes an orientation indicator that indicates how a medical device should be oriented on and/or inserted in the body of patient 18, e.g., when implanting the medical device. In the example of FIG. 16, template 200 includes orientation indicator 210 that indicates an orientation (e.g., a direction) in which IMD 12 should be implanted through an incision in the skin of patient 18. Orientation indicator 210 may indicate a directional path that can be followed during implantation of IMD 12. In some examples, orientation indicator 210 may indicate a range of acceptable implant orientations, e.g., by including several lines through a central axis and arrows illustrating a range of acceptable orientations.

In some examples, medical device positioning indication 206 may also include depth markings (not shown on FIG. 16) that help guide a clinician to implant IMD 12 at a satisfactory depth under the skin of patient 18. Depth markings may include an incision depth number (e.g., a number in millimeters adjacent incision indicator 208) that indicates how deep a clinician should make an incision in patient 18. Depth markings may also include a series of implant depth numbers that increase relative to incision indicator 208, thereby guiding the clinician on an angle at which IMD 12 should be inserted into patient 18. Additionally or alternatively, template 200 may include one or more delivery angle indicators (e.g., numbers or figures) that indicate an angle at which a clinician should insert IMD 12 into the body of patient 18. Depth markings and/or delivery angle indicators may be based on an estimated fat thickness of patient 18 and may be calibrated so that IMD 12 is implanted over a deep muscle fascia under a fat layer.

In addition to or in lieu of incision indicator 208 and orientation indicator 210, medical device positioning indicator 206 of template 200 may include one or more general implant region indicators 212. A general implant region indicator may designate a region of patient 18 in which IMD 12 may generally be implanted. General implant region indicator 212 may correspond to an anatomical region of patient 18 which, when IMD 12 is implanted in the region, causes IMD 12 to exhibit suitable operational performance. For instance, in the example of FIG. 16, general implant region indicator 212 may correspond to an anatomical region of patient 18 which, when IMD 12 is implanted in the region, causes IMD 12 to effectively sense R-waves and/or R-R intervals of a heart of patient 18.

In some examples, orientation indicator 210 may be a specific area within general implant region indicator 212 which, when IMD 12 is implanted in the area designated by orientation indicator 210, causes IMD 12 to exhibit substantially optimal operational performance. In other examples, template 200 may include a plurality of orientation indicators within general implant region indicator 212 such as, e.g., a plurality of lines of constant orientation within general implant region indicator 212. Instead of providing a single orientation indicator 210, the plurality of lines of constant orientation may indicate a general optimal orientation for implanting IMD 12 within general implant region indicator 212.

To generate template 200, signal measurements can be collected from IMD 12 at a variety of different locations and orientations within a test patient. The signal measurements may correspond to one or more physiological parameters of interest. The signal measurement process can then be repeated on different subjects that exhibit a range of different body types, were the range of body types corresponds to a range of body types in which it is expected that IMD 12 may subsequently be used. Subsequently, signal and/or statistical analysis techniques can be used to determine a best location, orientation, and/or region for implanting IMD 12 so that the IMD generate an optimal signal during use.

Template 200 can be implemented using a variety of different materials. In some examples, template 200 may be fabricated from a clear or semi-opaque material, e.g., so that a clinician can see through the material to align the template on the body of patient 18. In such examples, various indicia on template 200 may be formed in bright or dark colors to make the indicia easy to see against the clear or semi-opaque background. Template 200 may, but need not, include an adhesive region on a portion of the template that is placed against a patient's skin to prevent the template from moving, e.g., while implanting IMD 12.

In some examples, template 200 may have apertures (e.g., holes or slots) extending through the template to simplify the positioning of IMD 12 (e.g., above or below the skin of patient 18, depending on the configuration of IMD 12). In one example, template 200 includes an aperture that matches a profile of one or more sensors to be mounted to patient 18. A clinician can use the aperture to mark a location on the skin of patient 18, or, in other examples, the clinician may place a sensor directly within and in alignment with the aperture. Alternatively, a clinician may make an incision through the skin of patient 18 in an area exposed through the aperture. IMD 12 can then be placed under the skin of patient 18 (e.g., through the aperture) in alignment with markings on template 200.

While template 200 is generally described above as being configured to guide a clinician to implant IMD 12, it should be appreciated that template 200 may be configured to position any type of medical device on or in the body of a patient. Examples of medical devices that may benefit from an anatomical template position indicator include, but are not limited to, miniature ECG sensors with closely spaced electrodes, microphones for detecting heart sounds, and heart or blood flow measurement systems such as, e.g., ultrasound sensors.

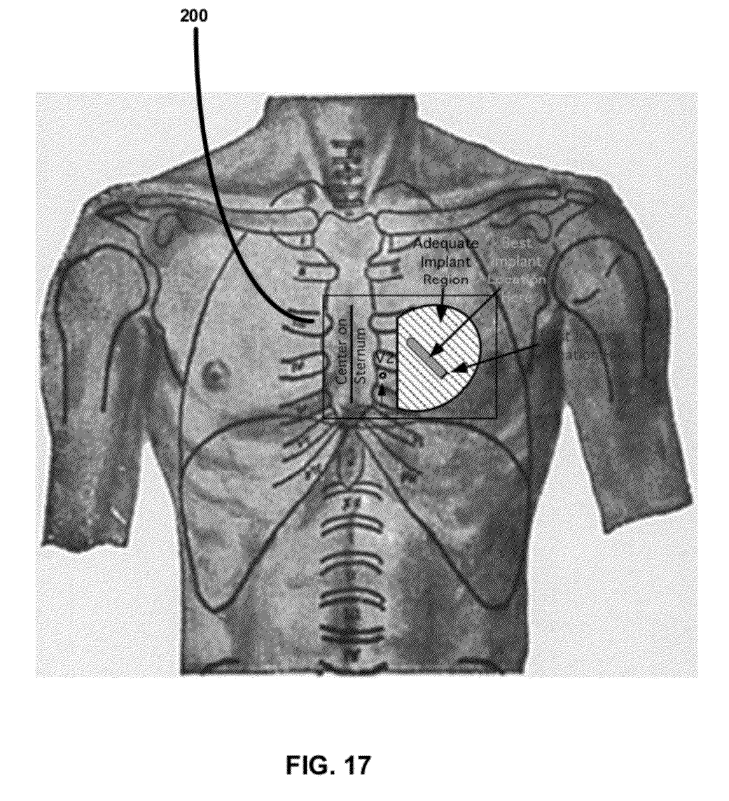

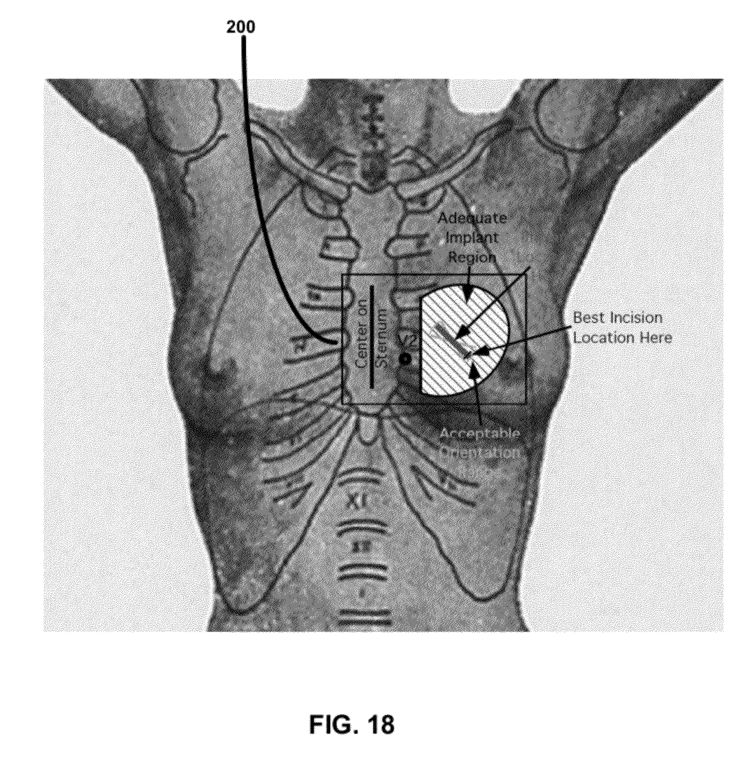

FIG. 17 is schematic drawings illustrating an example of template 200 attached to a male patient. FIG. 18 is a schematic drawings illustrating template 200 attached to a female patient.

Various modifications of the illustrative examples, as well as additional examples consistent with the disclosure, will be apparent to persons skilled in the art upon reference to this description.

1. A percutaneous delivery tool comprising:
a handle extending from a proximal end to a distal end;
a first attachment member;
a second attachment member;
a first connecting member connecting the first attachment member to the handle; and
a second connecting member connecting the second attachment member to the handle,
wherein the first attachment member and the second attachment member are configured to receive a medical device such that the medical device is positioned between the first attachment member and the second attachment member,
wherein at least one of the first attachment member and the second attachment member is movable relative to the other of the first attachment member and the second attachment member to releasably attach the medical device to the handle, and
wherein the first connecting member and the second connecting member are both angled with respect to the handle, the first connecting member is angled with respect to the first attachment member, and the second connecting member is angled with respect to the second attachment member.

2. The percutaneous delivery tool of claim 1, wherein the first attachment member defines a first major length, the second attachment member defines a second major length, and the first and second major lengths are both substantially parallel to each other.

3. The percutaneous delivery tool of claim 1, wherein the handle comprises a first handle member connected to the first connecting member, and a second handle member connected to the second connecting member.

4. The percutaneous delivery tool of claim 1, wherein the first and second connecting members define a first connecting angle between approximately ten and approximately eighty degrees between the handle and the first and second connecting members, and the first and second connecting members define a second connecting angle between approximately ten degrees and approximately eighty degrees between the first and second connecting members and the first and second attachment members.

5. The percutaneous delivery tool of claim 1, wherein the medical device has an elongated body defining a proximal end and a distal end, and the first attachment member and the second attachment member are configured to receive the medical device without substantially enclosing either the proximal end or the distal end of the medical device.

6. The percutaneous delivery tool of claim 1, wherein at least one of the first attachment member and the second attachment member defines a substantially planar surface that is configured to be positioned against a surface of the medical device.

7. The percutaneous delivery tool of claim 1, wherein the first attachment member and the second attachment member are configured to receive the medical device such that at least a distal half of the medical device projects away from a distal end of the first and second attachment members.

8. The percutaneous delivery tool of claim 1, wherein at least one of the first attachment member and the second attachment member is biased toward the other of the first attachment member and the second attachment member.

9. The percutaneous delivery tool of claim 1, further comprising a locking mechanism, wherein the locking mechanism is configured, when the medical device is positioned between the first attachment member and the second attachment member, to hold at least one of the first attachment member and the second attachment member in a fixed position relative to the other of the first attachment member and the second attachment member, and wherein the locking mechanism is releasable to allow at least one of the first attachment member and the second attachment member to move relative to the other of the first attachment member and the second attachment member.

10. A system comprising:
a medical device having an elongated body that defines a proximal end and a distal end; and
a percutaneous delivery tool comprising:
a handle extending from a proximal end to a distal end;
a first attachment member;
a second attachment member;
a first connecting member connecting the first attachment member to the handle; and
a second connecting member connecting the second attachment member to the handle,
wherein the medical device is positioned between the first attachment member and the second attachment member,
wherein at least one of the first attachment member and the second attachment member is movable relative to the other of the first attachment member and the second attachment member to releasably hold the medical device between the first attachment member and the second attachment member, and
wherein the first connecting member and the second connecting member are both angled with respect to the handle, the first connecting member is angled with respect to the first attachment member, and the second connecting member is angled with respect to the second attachment member.

11. The system of claim 10, wherein the first attachment member defines a first major length, the second attachment member defines a second major length, and the first and second major lengths are both substantially parallel to each other.

12. The system of claim 10, wherein the handle comprises a first handle member connected to the first connecting member, and a second handle member connected to the second connecting member.

13. The system of claim 10, wherein the first and second connecting members define a first connecting angle between approximately ten degrees and approximately eighty degrees between the handle and the first and second connecting members, and the first and second connecting members define a second connecting angle between approximately ten degrees and approximately eighty degrees between the first and second connecting members and the first and second attachment members.

14. The system of claim 10, wherein the medical device includes two electrode areas located at substantially opposite ends of the elongated body, and wherein the first attachment member and the second attachment member are disposed adjacent the medical device without substantially enclosing either of the two electrode areas.

15. The system of claim 10, wherein the first attachment member and the second attachment member each define substantially planar surfaces disposed adjacent the elongated body of the medical device, and wherein the first attachment member and the second attachment member each extend substantially parallel to a major axis defined by the medical device.

16. The system of claim 10, wherein at least a distal half of the medical device projects away from a distal end of the first and second attachment members.

17. The system of claim 10, wherein at least one of the first attachment member and the second attachment member is biased toward the other of the first attachment member and the second attachment member.

18. The percutaneous delivery tool of claim 1, further comprising a locking mechanism that holds at least one of the first attachment member and the second attachment member in a fixed position relative to the other of the first attachment member and the second attachment member, wherein the locking mechanism is releasable to allow at least one of the first attachment member and the second attachment member to move relative to the other of the first attachment member and the second attachment member.

19. A method comprising:
inserting a medical device having an elongated body defining a proximal end and a distal end into a patient, and
releasing the medical device from a percutaneous delivery tool to which the medical device is attached,
wherein the percutaneous delivery tool comprises:
a handle extending from a proximal end to a distal end;
a first attachment member;
a second attachment member;
a first connecting member connecting the first attachment member to the handle; and
a second connecting member connecting the second attachment member to the handle,
wherein the medical device is positioned between the first attachment member and the second attachment member prior to being released,
wherein at least one of the first attachment member and the second attachment member is movable relative to the other of the first attachment member and the second attachment member to releasably hold the medical device between the first attachment member and the second attachment member, and
wherein the first connecting member and the second connecting member are both angled with respect to the handle, the first connecting member is angled with respect to the first attachment member, and the second connecting member is angled with respect to the second attachment member.

20. The method of claim 19, wherein the handle comprises a first handle member connected to the first connecting member, and a second handle member connected to the second connecting member.

21. The method of claim 20, wherein the first and second connecting members define a first connecting angle between approximately ten degrees and approximately eighty degrees between the handle and the first and second connecting members, and the first and second connecting members define a second connecting angle between approximately ten degrees and approximately eighty degrees between the first and second connecting members and the first and second attachment members.

22. The method of claim 20, wherein inserting the medical device comprises inserting the distal end of the medical device through an incision in the skin of the patient, and rotating the percutaneous delivery tool to place the elongated body of the medical device substantially parallel to the skin of the patient.

23. The method of claim 20, wherein the medical device includes two electrode areas located at substantially opposite ends of the elongated body, and wherein the first attachment member and the second attachment member are disposed adjacent the medical device without substantially enclosing either of the two electrode areas.

24. The method of claim 20, further comprising sensing an electrical signal after inserting the medical device into the patient, and repositioning the medical device in the patient based on the sensed electrical signal before releasing the medical device.

25. The method of claim 20, wherein the first attachment member and the second attachment member each define substantially planar surfaces disposed adjacent the elongated body of the medical device, and wherein the first attachment member and the second attachment member each extend substantially parallel to a major axis defined by the medical device.

26. The method of claim 20, wherein the percutaneous delivery tool further comprises a locking mechanism that holds at least one of the first attachment member and the second attachment member in a fixed position relative to the other of the first attachment member and the second attachment member, and wherein releasing the medical device comprises releasing the locking mechanism so that at least one of the first attachment member and the second attachment member is movable relative to the other of the first attachment member and the second attachment member.

27. A percutaneous delivery tool comprising:
an elongated body extending from a proximal end to a distal end, the elongated body including a substantially straight proximal portion, a substantially straight intermediate portion, and a substantially straight distal portion,
wherein the substantially straight proximal portion is angled with respect to the substantially straight intermediate portion, and the substantially straight distal portion is angled with respect to the substantially straight intermediate portion, and
wherein the substantially straight distal portion is configured to receive a medial device such that a distal portion of the medical device extends beyond the substantially straight distal portion of the elongated body.

29. The percutaneous delivery tool of claim 27, wherein the substantially straight distal portion defines a cavity that is configured to receive a proximal portion of the medial device.

30. The percutaneous delivery tool of claim 27, further comprising a release mechanism configured to medical device from the delivery tool.

31. The percutaneous delivery tool of claim 27, wherein the substantially straight proximal portion and the substantially straight intermediate portion define a first connecting angle between approximately ten and approximately eighty degrees, and the substantially straight intermediate portion and the substantially straight distal portion define a second connecting angle between approximately ten degrees and approximately eighty degrees.
